(19) United States
(12) Patent Application Publication
(10) Pub. No.: US 2013/0034863 A1
(43) Pub. Date: Feb. 7, 2013

(54) Apparatus and Methods for Detecting Inflammation Using Quantum Dots

(71) Applicants: Elisabeth S. Papazoglou, Yardley, PA (US); Sreekant Murthy, Sewell, NJ (US); Sundar Babu Nadarajan, Ashburn, VA (US); Sakya Sing Mohapatra, Lansdale, PA (US)

(72) Inventors: Elisabeth S. Papazoglou, Yardley, PA (US); Sreekant Murthy, Sewell, NJ (US); Sundar Babu Nadarajan, Ashburn, VA (US); Sakya Sing Mohapatra, Lansdale, PA (US)

(73) Assignees: PHILADELPHIA HEALTH AND EDUCATION CORPORATION; DREXEL UNIVERSITY

(21) Appl. No.: 13/145,950

(22) PCT Filed: Jan. 22, 2010
(86) PCT No.: PCT/US10/21821

§ 371 Date: Feb. 10, 2012

Related U.S. Application Data
(60) Provisional application No. 61/146,928, filed on Jan. 23, 2009.

(51) Int. Cl. G01N 21/64; C09K 11/08; C12M 1/42; C09K 11/88; B82Y 15/00

(52) U.S. Cl. 435/7.4; 435/287.2; 436/501; 422/69; 252/301.6 S; 252/301.36; 252/301.4 R; 977/774; 977/920

(57) ABSTRACT

Apparatus and methods for detecting an a biomarker indicative of an inflammatory condition, including a capillary tube adapted for one or more biomarkers to adhere to an interior surface thereof, a light source for energizing quantum dots conjugated with the biomarkers within the capillary tube, and a detection system for detecting and quantifying fluorescent energy emitted by the quantum dots in one or more predetermined wavelength ranges, each wavelength range being correlated to one and only one of the biomarkers. A method of stabilizing the fluorescence intensity of quantum dots is also disclosed.

### CROSS-REFERENCE TO RELATED APPLICATION

This application is a U.S. national phase application filed under 35 U.S.C. §371 claiming benefit to International Patent Application No. PCT/US2010/021821, filed on Jan. 22, 2010, which is entitled to priority under 35 U.S.C. §119(e) to U.S. Provisional Patent Application No. 61/146,928, filed on Jan. 23, 2009, each of which application is hereby incorporated herein by reference in its entirety.

### BACKGROUND

Inflammatory bowel disease (IBD) encompasses two chronic, related inflammatory conditions, ulcerative colitis (UC) and Crohn's disease (CD). In addition, organs other than the intestinal tract can be involved by the underlying inflammation of IBD thus making IBD a multi-organ disease. As many as 4 million people (including one million Americans) worldwide suffer from a form of IBD. In the U.S. alone, MD accounts for approximately 152,000 hospitalizations each year. The annual medical cost for the care of IBD patients in the United States is estimated at over $2 billion. When adjusted for loss of productivity, the total economic burden is estimated to be nearly $3 billion.

Inflammatory bowel disease is a complex, multifactorial sequelae characterized by severe derangements in the structure and function of local tissue architecture and increased presence of neutrophils and lymphocytes and other pro-inflammatory cells. In addition, epithelial, endothelial, mesenchymal, adipose tissue and nerve cells all can exhibit a broad range of damage as a result of the inflammatory process. Effector, regulatory and immune-like functions interact abnormally with lymphoid cells to further contribute to the pathogenesis of inflammatory disease. Heart disease, arthritis, asthma, allergy, infection and diabetes all have elements of chronic inflammation. Examples of inflammatory disease also include, but are not limited to, stroke, cardiovascular disease, acute coronary syndromes, acute myocardial infarction, pericarditis, periodontal disease, cancer, Alzheimer's disease, and inflammatory bowel disease. Inflammatory disease can also affect multiple organ systems, as in autoimmune diseases.

Inflammation is a significant contributor to the pathogenesis of both the acute and chronic stages of IBD. The diagnosis of IBD is rarely straightforward, involving an extensive process of examination and invasive testing, including biopsy during endoscopy. Even with these specialized studies, it is often still difficult to tell which type of IBD a person has, leading to a diagnosis of “indeterminate colitis” and rendering disease management more difficult. UC carries a significant risk for the development of colorectal cancer, but remains difficult to differentiate from CD. Since UC in particular is associated with a 35% higher risk of developing colorectal cancer than the general population, making a proper diagnosis is essential to good patient care.

While there is no medical cure for IBD, effective medical treatment is available which can calm the inflammation and relieve the symptoms of diarrhea, abdominal pain, and rectal bleeding. Since the disease tends to manifest itself with multiple attacks and remissions, continuous monitoring of patients is essential to provide the necessary medical treatment to reduce inflammation and prevent the development of clinical sequelae.

The current noninvasive tests which are used clinically to distinguish between UC from CD, are based on the presence of antibodies such as perinuclear antineutrophilic cytoplasmic antibody (p-ANCA) and anti-Saccharomyces cerevisiae antibody (ASCA) in serum, and have less than 70% specificity. Mostly invasive biopsies are used to confirm presence of a particular disease.

Therefore, there is an unmet need for a technology to quantify inflammatory markers quickly, cost effectively, and with high sensitivity. Such quantification could lead not only to differential diagnosis but also to the evaluation of response to therapy in inflammatory diseases. More specifically, there is a long-standing need for non-invasive diagnostic tools that are able to distinguish non-IBD symptoms from IBD, accurately distinguish UC from CD, and monitor disease progression, remission or relapse. In particular, there is a need for a technology to determine low levels of inflammatory markers and to utilize the ability to detect these markers as predicators of inflammatory diseases, responses to therapy for inflammatory diseases, and progressions to cancer.

Additionally, there is a need to improve the stability of fluorescence intensity emitted from quantum dots over time as the quantum dots during storage or diagnostic imaging, so as to avoid the loss of valuable information.

### SUMMARY

The present invention includes an apparatus for detecting a biomarker indicative of an inflammatory condition. The apparatus has a capillary tube adapted for one or more biomarkers to adhere to an interior surface thereof, a light source for energizing quantum dots conjugated with the biomarkers within the capillary tube, and a detection system for detecting and quantifying fluorescent energy emitted by the quantum dots in one or more predetermined wavelength ranges, each wavelength range being correlated to one and only one of the biomarkers.

In one embodiment of the apparatus, the capillary tube comprises at least one material selected from the group of polymethyl methacrylate (PMMA), polyvinyl acetate, and polystyrene tube. A hypodermic needle can be connected to an end of the capillary tube for supplying a sample to the capillary tube. The capillary tube can be supported externally by a glass capillary tube.

In another embodiment, the apparatus also includes a fluid handling unit adapted to hold multiple capillary tubes and a mechanical positioning system for successively positioning each capillary tube to enable the sample contained therein to be exposed to the light source and visible to the detection system.

In another embodiment, the capillary tube has a volume in the range of about 100 nanoliters to about 1 microliter.

In another embodiment of the apparatus, the light source comprises an LED, a laser diode, or an array having a plurality of LEDs or laser diodes. One or more of the LEDs can be ultraviolet LEDs. The light source can further comprise a lens for focusing the LED or LEDs onto the capillary tube.

In another embodiment of the apparatus, the detection system comprises a broadband filter. In a still another embodiment, the detection system comprises a photodetector. The photodetector can be a spectrometer coupled to at least one photomultiplier tube, avalanche photodiode detector, or a CCD camera. The detection system can include a mirror disposed around at least a portion of the capillary tube for increasing the amount of the fluorescent energy emitted by the quantum dots that can be detected by the photodetector. The mirror can be selected from the group of a spherical mirror, a cylindrical mirror, and a parabolic mirror.

In another embodiment of the apparatus, the capillary tube is a polymethyl methacrylate (PMMA) capillary tube having a volume of less than about 1.5 microliters, wherein the light source comprises an ultraviolet LED, and wherein the detection system comprises a CCD camera. In yet another embodiment, the detection system comprises a spherical mirror for focusing energy emitted by the quantum dots to the CCD camera. In one variation, a first LED of the light source is directed into an end of the capillary tube and the CCD camera detects energy emitted through the wall of the capillary tube. Additionally, a second LED of the light source can be directed into an opposite end of the capillary tube. In another variation, at least one LED of the light source is disposed adjacent to a wall of the capillary tube and the CCD camera detects energy emitted through an end of the capillary tube.

In another embodiment of the apparatus, said biomarker is selected from the group consisting of myeloperoxidase (MPO), IL-1α, TNFα, perinuclear anti-neutrophil cytoplasmic antibody (p-ANCA), anti-Saccharomyces cerevisiae antibody (ASCA), angiotensin converting enzyme, lactoferrin, C-reactive protein (CRP), and calprotectin.

In another embodiment, the apparatus further comprises a composition for detecting a biomarker in a biological sample contained in the capillary tube, wherein said composition comprises at least one conjugate comprising a quantum dot and an antibody that specifically binds to a biornarker. The antibody can be bound to a substrate surface.

The present invention also includes a method of diagnosing an inflammatory condition in a subject by detecting a biomarker in a sample. The method includes providing a sample to a capillary tube coated with an antibody, the sample potentially including a biomarker indicative of the inflammatory condition, contacting the sample with a conjugate comprising a quantum dot and an antibody that specifically binds to the biomarker, energizing the quantum dot with a light source, detecting fluorescent emission from the quantum dot, and correlating the fluorescent emission to the concentration of the biomarker in the sample.

In one embodiment of the diagnostic method, said biomarker is selected from the group consisting of an enzyme, an adhesion molecule, a cytokine, a protein, a lipid mediator, an immune response mediator, and a growth factor.

In another embodiment of the method, said biomarker is selected from the group consisting of myeloperoxidase (MPO), IL-1α, TNFα, perinuclear anti-neutrophil cytoplasmic antibody (p-ANCA), anti-Saccharomyces cerevisiae antibody (ASCA), angiotensin converting enzyme, lactoferrin, C-reactive protein (CRP), and calprotectin.

In on embodiment, the capillary tube is functionalized using NaOH. Alternatively, the capillary tube is functionalized using plasma or ultraviolet light.

In another embodiment of the diagnostic method, said inflammatory condition comprises at least one inflammatory disease selected from the group consisting of inflammatory bowel disease, ulcerative colitis, Crohn's disease, stroke, myocarditis, cardiovascular disease, acute coronary syndromes, acute myocardial infarction, pericarditis, periodontal disease, cancer, Alzheimer's disease, and autoimmune diseases.

The invention further includes a method of stabilizing the fluorescence of quantum dots over time comprising exposing the quantum dots to a fluorescence stabilizing medium.

In one embodiment of the stabilization method, the fluorescence stabilizing medium is a solution having a low ionic strength.

In other embodiments of the stabilization method, the fluorescence stabilizing medium is a solution having a pH greater than or equal to about 7.0, or a pH greater than or equal to about 8.0.

In another embodiment of the stabilization method, the fluorescence stabilizing medium comprises water-soluble free radical quenchers. In still another embodiment, the fluorescence stabilizing medium comprises TrisPro and an amount of water-soluble vitamin E. In one variation, the amount of vitamin E is at least about 0.001% of the medium.

In another embodiment, the quantum dots each comprise a CdSe core and a ZnS protective layer.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 is a schematic depicting an apparatus for detecting quantum dot (QD) fluorescence from a sample in waveguide mode.

FIG. 2 is an overview flow chart of a method for detecting and diagnosing inflammatory bowel disease.

FIG. 3 is a schematic depicting a process for binding QD conjugates to antigens.

FIG. 4 is a schematic depicting a sandwich Quantum-Linked ImmunoSorbent Assay (QLISA) process.

FIG. 5 is a schematic depicting a competitive QLISA process.

FIGS. 6A and 6B are schematics depicting an apparatus for detecting fluorescence form a QD-linked sample in waveguide mode.

FIG. 7 is a photograph of the apparatus of FIG. 6B in use.

FIG. 8 is a schematic depicting an apparatus for causing a sample to fluoresce by exposure to LED light sources in side illumination mode.

FIG. 9 is a schematic depicting an apparatus for causing a sample to fluoresce by exposure to focused LED light sources in side illumination mode.

FIG. 10 is a schematic depicting the conversion of raw image data from an apparatus as in FIG. 8 or FIG. 9 into a processed image from which intensity information can be obtained.

FIGS. 11A through 11D are schematics depicting alternate apparatuses for detecting fluorescence from a QD-linked sample.

FIG. 12 is a graph correlating concentration of a biomarker (MPO) in a sample with intensity of fluorescence emissions.

FIG. 13 is a graph correlating the concentration of MPO with intensity of fluorescence emissions to determine a detection threshold.

FIG. 14 is graph correlating the concentration of MPO with intensity of fluorescence emissions in an animal sample as compared with a control sample.

FIG. 15 is a process flow chart of a diagnostic protocol in an embodiment of the present invention.

FIGS. 16A and 19B are schematic illustrations of configurations of apparatus for collecting fluorescent signals from QDs in a PMMA capillary.

FIGS. 17A through 17F are optical micrographs of capillaries demonstrating the effect of blocking.

FIGS. 18A and 18B are optical micrographs of PMMA capillaries treated with DB-Ab to demonstrate the effect of surfactant in the wash buffer.

FIGS. 19A and 19B show the sensitivity of an embodiment of the QLISA method of the present invention to MPO and representative images of a PMMA capillary comparing a sample of 0.3 nM MPO with a control.

FIG. 20 is a comparison of fluorescence intensities of QD solutions obtained with side illumination and waveguide configurations of the apparatus of the present invention.

FIGS. 21A through 21C compare the fluorescence intensity at various MPO concentrations obtained by side illumination.

FIG. 22 shows the fluorescence intensity from MPO-spiked animal stool samples.

FIG. 23 is a graph showing interference from non-specific binding to MMP-13.

FIG. 24 is a chart comparing disease time line and fluorescence intensity of MPO bound to quantum dots.

FIG. 25 is a schematic depicting a fluid handling unit for multiple PMMA tubes.

FIG. 26 is a photograph showing an exemplary fluid handling unit for multiple PMMA tubes.

FIG. 27 is a schematic showing the designation of reservoirs for use with a fluid handling unit as in FIG. 25 or FIG. 26.

FIG. 28A to 28D are graphics depicting the fabrication of a multiple sample holder using a mold.

FIGS. 29A and 29B are comparisons showing the effect of storage buffer on the fluorescence intensity of QDs and on QD stability over a period of time.

FIG. 30 shows the fluorescence intensity from lactoferrin-spiked human stool samples.

FIG. 31 is a schematic of an embodiment of a sampling manifold.

FIG. 32 is a schematic of an embodiment of a detachable multiple capillary holder.

FIGS. 33A and 33B show the molecular structure of a buffer medium and a water-soluble vitamin E used to stabilize QD fluorescence intensity.

FIGS. 34A and 34B are graphs comparing the decay in fluorescence intensity of QDs over time as a function of pH, in solutions without and with vitamin E, respectively.

FIGS. 35A and 35B are graphs comparing the decay in fluorescence intensity of QDs over time as function of pH, in solutions without and with vitamin E, respectively.

FIGS. 36A and 36B are graphs comparing the decay in fluorescence intensity of QDs over time as a function of vitamin E concentration at 6.5 pH, for Ocean Nanotech QDs and Invitrogen QDs, respectively.

FIGS. 37A and 37B are graphs comparing the decay in fluorescence intensity of QDs over time as a function of vitamin E concentration at 7.5 pH, for Ocean Nanotech QDs and Invitrogen QDs, respectively.

FIGS. 38A and 38B are graphs comparing the decay in fluorescence intensity of QDs over time as a function of vitamin E concentration at 8.5 pH, for Ocean Nanotech QDs and Invitrogen QDs, respectively.

### DETAILED DESCRIPTION

The present invention discloses the development of a simple and inexpensive quantum dot based immunoassay for detecting myeloperoxidase (MPO) in biological samples is reported. The acronym QLISA is introduced to represent Quantum-Linked ImmunoSorbent Assay. In a preferred embodiment, the detection method utilizes polymethylmethacrylate (PMMA) micro-capillaries as substrate for performing a sandwich assay. UV-LEDs both high power (80 mW) and low power (10 mW) were tested for their efficiency in maximizing detection sensitivity in either a waveguide illumination or a side illumination mode. The results discussed herein indicate that both waveguide and side illumination modes can be employed for detecting MPO down to 15 ng/ml; however, using the high power LED in a side illumination mode unexpectedly improves sensitivity and simplifies the data acquisition. A testing protocol and robustness of embodiments of sensors were evaluated with animal stool samples spiked with MPO and the results indicate that the sensitivity of the capture and reporter antibodies is not compromised when used in stool samples. Further, the effect of the ionic strength of the environment on the fluorescence stability of quantum dots was evaluated and found to affect the assay, particularly if long imaging times are necessary. Notably, replacing the buffer with glycerol or another non-polar or weakly polar substance during imaging increased the fluorescence intensity of quantum dots while significantly minimizing the loss in intensity, even after relatively short times of two hours.

In general, the invention is directed to an apparatus and a method for detecting and quantifying biomarkers indicative of inflammatory disease, particularly inflammatory bowel disease, with sufficient sensitivity to distinguish non-MD symptoms from IBD symptoms and to differentiate UC from CD. An apparatus according to the invention holds a sample in which one or more biomarkers have been conjugated with quantum dots and provides a light source for energizing the quantum dots and a detection system for detecting and quantifying the quantum dots. A method of using an apparatus according to the invention includes collecting a sample, conjugating quantum dots to biomarkers in the sample, energizing the quantum dots with a light source, detecting emission from the quantum dots, and determining the concentration of the biomarker in the sample based on a correlation between detected emission and biomarker concentration.

In one embodiment, an apparatus is provided for detecting a biomarker indicative of an inflammatory condition. The apparatus includes a capillary tube adapted for one or more biomarkers to adhere to an interior surface thereof. The apparatus further includes a light source for energizing quantum dots conjugated with the biomarkers within the capillary tube and a detection system for detecting and quantifying fluorescent energy emitted by the quantum dots in one or more predetermined wavelength ranges, each wavelength range being correlated to one and only one of the biomarkers.

In another embodiment, a method is provided for diagnosing an inflammatory condition by detecting a biomarker in a sample. The method includes providing a sample to a capillary tube coated with an antibody, the sample potentially including a biomarker indicative of the inflammatory condition. The method further includes contacting the sample with a conjugate comprising a quantum dot and an antibody that specifically binds to the biomarker, energizing the quantum dot with a light source, detecting fluorescent emission from the quantum dot, and correlating the fluorescent emission to the concentration of the biomarker in the sample.

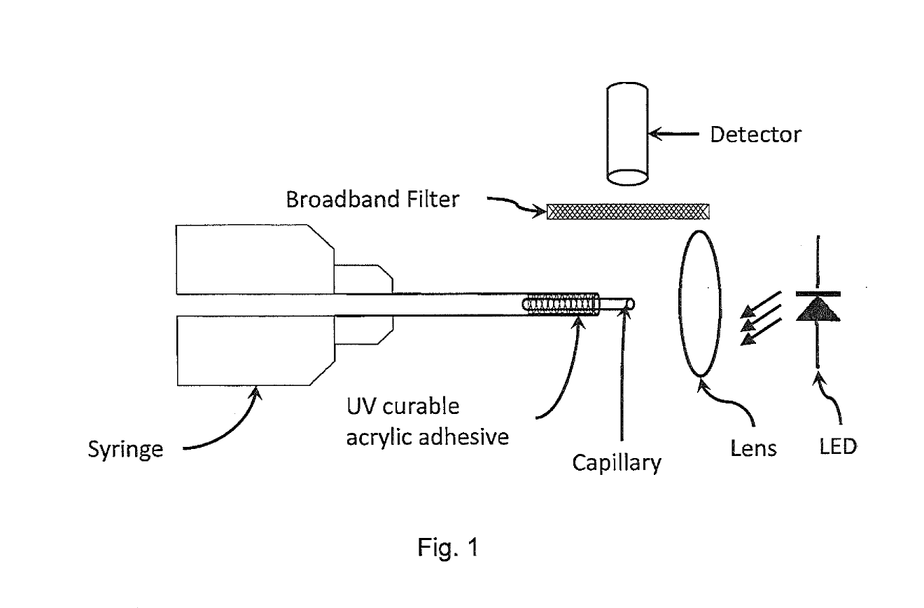

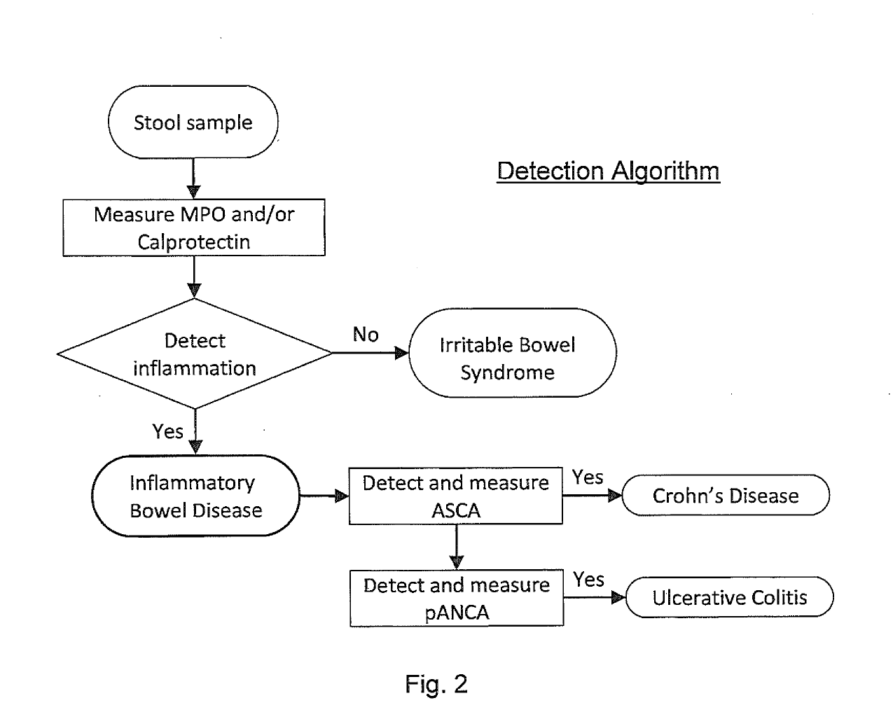

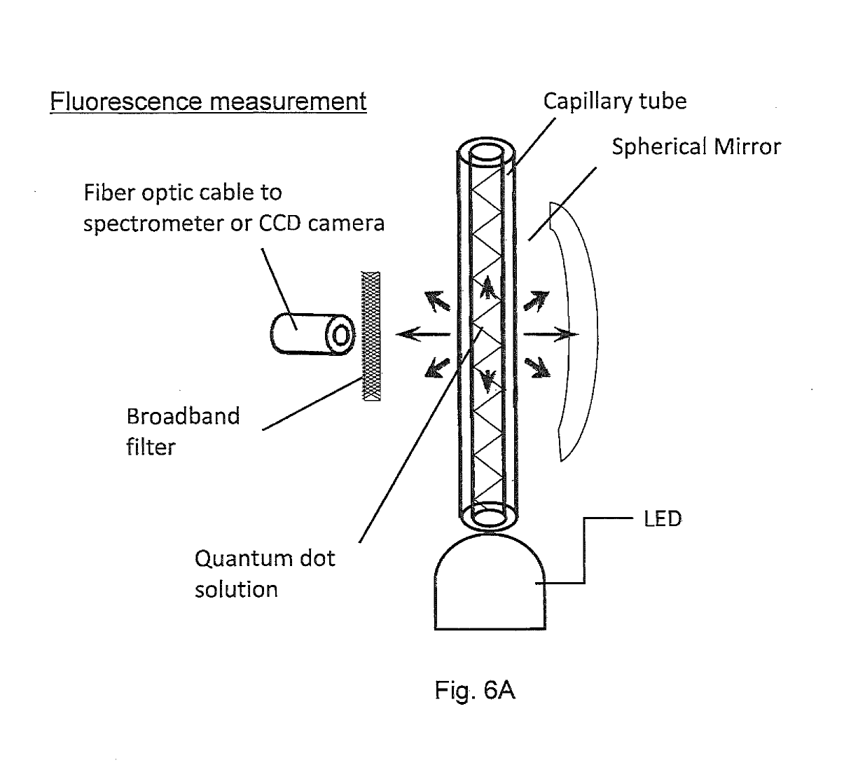

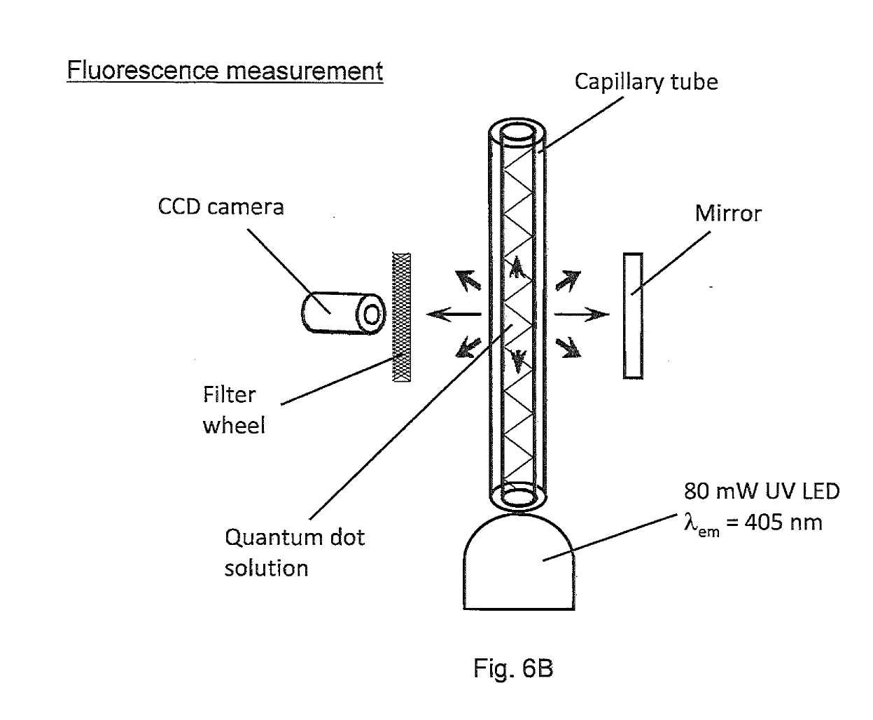

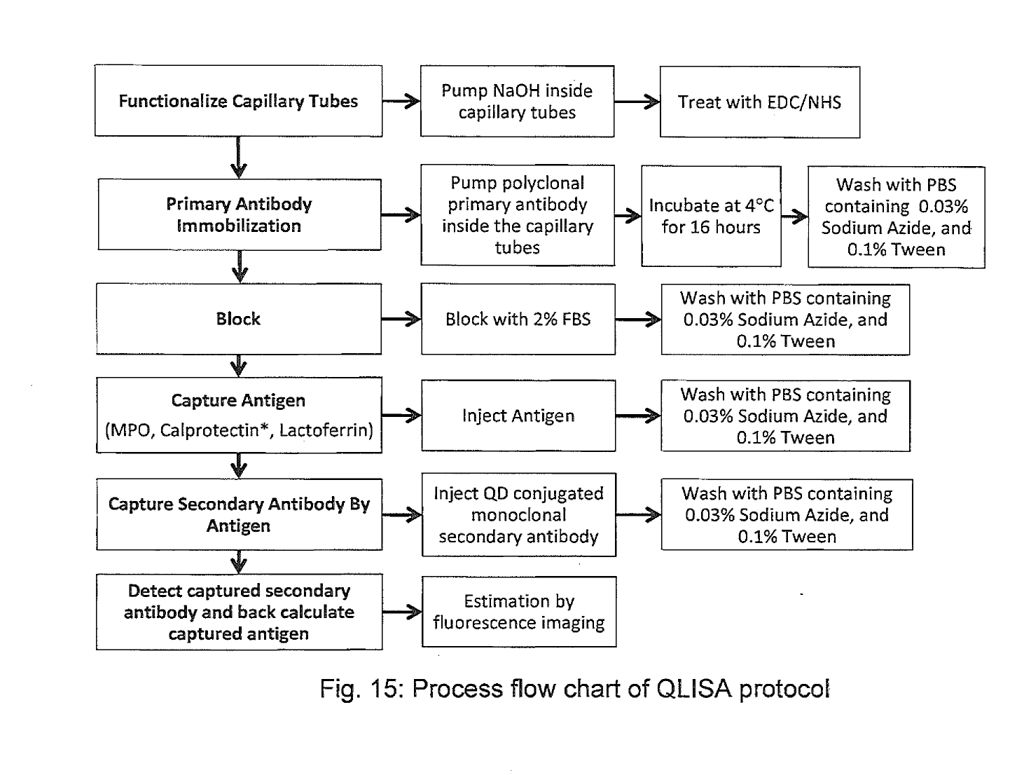

There is shown in FIG. 1 an embodiment of an apparatus for detecting QD fluorescence from a sample as part of a method for rapidly identifying and measuring biochemical and immunological markers for inflammatory disease. An apparatus such as that shown in FIG. 1, or alternatively apparatuses as shown in FIGS. 6A-9, 11A-11D, and 16A-16B can be used in a diagnostic method as depicted generally in FIG. 2 and more specifically in FIG. 15 for detecting the presence of inflammatory diseases including but not limited to inflammatory bowel disease (IBD), and, for example, for diagnosing whether the IBD is characterized by ulcerative colitis (UC) or Crohn's disease (CD).

Methods of quantitatively assessing inflammation with biosensing nanoparticles are described in detail in commonly assigned PCT Application No. PCT/US2007/015748 filed on Jul. 11, 2007, and incorporated by reference herein in its entirety. The nanoparticles include quantum dots conjugated to targeting moieties that specifically bind to a biomarker protein or a nucleic acid encoding a biomarker, where dysregulation of the biomarker is associated with inflammatory disease.

In particular, methods disclosed herein use monoclonal antibodies conjugated to quantum dots as a means of detecting nanolevels of biomarkers. As used herein, the methods have collectively been dubbed “Quantum-Linked ImmunoSorbent Assay” (QLISA) as differentiated from the technique known in the art as Enzyme-Linked ImmunoSorbent Assay (ELISA). QLISA possesses advantages over ELISA, as will be described herein.

Any of the apparatus described herein can be provided as a test kit comprising a single assay with customized antibody coated micro-columns and ready-to-use reagents for rapid and easy detection. The assay may comprise MPO, IL-1α, TNFα, calprotectin, lactoferrin, fibronectin, ASCAm p-ANCA, and/or other markers, particularly those that may be found in fecal samples as indicators of various forms of IBD. The apparatus and methods can be adapted to detect, at pico- or nano-molar concentrations, single markers in sequence or multiple markers simultaneously. The test kit is adapted to measure inflammatory biomarkers in biological samples (e.g., fluids and fecal samples) using QLISA, i.e., quantum dot immobilization and fluorescence. The test kit can be used in a physician's office as a point of care screening device, or as part of a battery of tests done at a diagnostics laboratory.

The present approach is based on using a combination of available biomarkers (Myeloperoxidase-MPO, p-ANCA, ASCA) to lead to differential diagnosis of Inflammatory Bowel Disease (IBD) from Inflammatory Bowel Syndrome (IBS) and to differentiate Ulcerative Collitis (UC) from Crohn's Disease (CD).

### DEFINITIONS

As used herein, each of the following terms has the meaning associated with it in this section.

The articles “a” and “an” refer to one or to more than one (i.e. to at least one) of the grammatical object of the article. By way of example, “an element” means one element or more than one element.

The terms “about” and “approximately” will be understood by persons of ordinary skill in the art and will vary to some extent on the context in which it is used.

The term “antibody” refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, intracellular antibodies (“intrabodies”), Fv, Fab and F(ab)₂, as well as single chain antibodies (scFv), camelid antibodies and humanized antibodies.

An “antigen” or “Ag” refers to a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

A “biological sample” refers to any sample comprising a cell, a tissue, or a bodily fluid obtained from an organism in which expression of a biomarker can be detected. An example of such a biological sample includes a “body sample” obtained from a human patient.

A “body sample” includes, but is not limited to blood, lymph, urine, gynecological fluids, biopsies, amniotic fluid, stool samples, fecal samples, and smears. Samples that are liquid in nature are referred to herein as “bodily fluids.” Body samples may be obtained from a patient by a variety of techniques including, for example, by scraping or swabbing an area or by using a needle to aspirate bodily fluids. Methods for collecting various body samples are well known in the art.

The term “dysregulation” refers to an over- or under-expression of a biomarker present and detected in a biological sample obtained from a putative at-risk individual, then compared with a biomarker in a sample obtained from one or more normal, not-at-risk individuals. In some instances, the level of biomarker expression is compared with an average value obtained from more than one not-at-risk individuals. In other instances, the level of biomarker expression is compared with a biomarker level assessed in a sample obtained from one normal, not-at-risk sample. In yet another instance, the level of biomarker expression in the putative at-risk individual is compared with the level of biomarker expression in a sample obtained from the same individual at a different time.

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence, Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “quantum dot” (QD) refers to a semiconductor nanostructure that confines the motion of conduction band electrons, valence band holes, or excitons (bound pairs of conduction band electrons and valence band holes) in all three spatial directions. The confinement can be due to electrostatic potentials (generated by external electrodes, doping, strain, impurities), the presence of an interface between different semiconductor materials (e.g. in core-shell nanocrystal systems), the presence of the semiconductor surface (e.g. semiconductor nanocrystal), or a combination of these. A quantum dot has a discrete quantized energy spectrum. The corresponding wave functions are spatially localized within the quantum dot, but extend over many periods of the crystal lattice. A quantum dot contains a small finite number (of the order of 1-100) of conduction band electrons, valence band holes, or excitons, i.e., a finite number of elementary electric charges. One of the optical features of small excitonic quantum dots immediately noticeable to the unaided eye is coloration. While the material which makes up a quantum dot defines its intrinsic energy signature, more significant in terms of coloration is the size. The larger the dot, the redder (the more towards the red end of the spectrum) the fluorescence. The smaller the dot, the bluer (the more towards the blue end) it is. The coloration is directly related to the energy levels of the quantum dot. Quantitatively speaking, the bandgap energy that determines the energy (and hence color) of the fluoresced light is inversely proportional to the square of the size of the quantum dot.

The term “conjugate” refers to a physical or chemical attachment of one molecule to a second molecule.

The term “specifically binds” refers to an action of a molecule, such as an antibody, which recognizes and binds to a cell surface molecule or feature, but does not substantially recognize or bind other molecules or features in a sample.

The term “variant” refers to a nucleic acid sequence or a peptide sequence that differs in sequence from a reference nucleic acid sequence or peptide sequence respectively, but retains essential properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

The term “inflammatory condition” refers generally to a continued presence of inflammation in a mammal past the initial, beneficial immune response. Inflammatory conditions include, but are not limited to, chronic wounds, arthritis, atherosclerosis, and inflammatory diseases, such as autoimmune diseases, stroke, cardiovascular disease, acute coronary syndromes, acute myocardial infarction, pericarditis, periodontal disease, cancer in terms of it's connection to inflammatory disease, Alzheimer's disease, and inflammatory bowel disease.

A “biomarker” refers to any gene, protein, or metabolite whose level of expression in a tissue, cell or bodily fluid is dysregulated compared to that of a normal or healthy cell, tissue, or biological fluid. In one embodiment, a biomarker to be measured according to the method of the invention selectively responds to the presence and progression of inflammatory disease in an individual.

By “selectively respond to the presence and progression of inflammatory disease” it is intended that the biomarker of interest is specifically over- or under-expressed in response to the onset and subsequent progression of inflammatory disease in an individual. This biomarker is not dysregulated during the course of other diseases, or other conditions not considered to be clinical disease. Thus, measuring the levels of biomarkers in the methods of the invention permits differentiation between samples collected from an individual with inflammatory disease and an individual without inflammatory disease.

### Biomarkers Correlated to Diseases

Specific biomarkers can be designed to be associated with specific diseases. A disease specific biomarker is a biomarker which is dysregulated in response to a particular disease but is not dysregulated during the course of other diseases or other conditions that are not considered clinical diseases. To make use of the disease specific association between a biomarker and a disease, an apparatus and method according to the invention can be used to detect a particular biomarker and the particular biomarker can be correlated with its respective associate disease, to indicate the presence of the disease. In particular, by using disease specific biomarkers associated with diseases such as IBD, UC, or CD, one of these specific inflammatory diseases can be detected.

In one embodiment, a biomarker to be measured selectively responds to the presence and progression of inflammatory disease in an individual, meaning that the biomarker of interest is specifically over- or under-expressed in response to the onset and subsequent progression of inflammatory disease in an individual. Measuring the levels of disease specific biomarkers in the methods disclosed herein permits differentiation between samples collected from an individual with inflammatory disease versus an individual without inflammatory disease, as well as an individual with UC versus an individual with CD.

In one aspect of the invention, the inflammatory bowel disease is ulcerative colitis. In another aspect of the invention, the inflammatory bowel disease is Crohn's disease. Further, by measuring the levels of the biomarkers in the method of the invention, a practitioner would be able to distinguish different forms of IBD, specifically UC from CD.

A biomarker that can be measured according to the invention includes proteins and variants and fragments thereof, that exhibit dysregulation during inflammatory disease. Biomarker nucleic acids useful in the invention should be considered to include both DNA and RNA comprising the entire or partial sequence of any of the nucleic acid sequences encoding the biomarker, or the complement of such a sequence. Similarly, a biomarker protein should be considered to comprise the entire or partial amino acid sequence of any of the biomarker proteins or polypeptides.

By way of a nonlimiting example, serological samples obtained from patients with IBD that are positive for perinuclear antineutrophil cytoplasmic antibody (pANCA) but negative for anti-Saccharomyces cerevisiae antibody (ASCA) are indicative of ulcerative colitis, while serological samples positive for ASCA but negative for pANCA are indicative of Crohn's disease. Biomarkers useful in the present invention include myeloperoxidase (MPO), IL-1α and TNFα. Other biomarkers useful in the present invention include, but are not limited to, perinuclear anti-neutrophil cytoplasmic antibody (p-ANCA), anti-Saccharomyces cerevisiae antibody (ASCA), angiotensin converting enzyme, lactoferrin, C-reactive protein, fibronectin, lactoferrin, and calprotectin. Additional biomarkers can include an enzyme, an adhesion molecule, a cytokine, a protein, a lipid mediator, and a growth factor.

In one embodiment, the biological activity of a biomarker of the invention is the ability of the biomarker to respond in a predictable way to the onset and progression of IBD. In one aspect, a biomarker responds to the onset and progression of UC. In another aspect, a biomarker responds to the onset and progression of CD.

Although a method of the invention requires the detection of at least one biomarker in a body sample, two or more biomarkers may be used to practice the method of the present invention. Therefore, in an embodiment, two or more biomarkers are used. In an aspect of the invention, two or more complementary biomarkers are used. Simultaneous detection of multiple biomarkers can be accomplished by conjugating differently sized quantum dots with different corresponding biomarkers such that each biomarker can be detected by a different wavelength emission associated with each size of the quantum dots.

When used to refer to a biomarker herein, the term “complementary” is intended to mean that detection of the combination of biomarkers in a body sample results in the successful identification of a patient with inflammatory disease in a greater percentage of cases than would be identified if only one biomarker was used. In one embodiment of the invention, two biomarkers may be used to more accurately identify a patient with IBD than when one biomarker is used. In one aspect of the invention, two or more biomarkers may be used to diagnose ulcerative colitis. In another aspect of the invention, two or more biomarkers are used to identify a patient with Crohn's disease.

Accordingly, where at least two biomarkers are used, at least two antibodies directed to distinct biomarker proteins will be used to practice the immunocytochemistry methods disclosed herein. The antibodies may be contacted with the body sample simultaneously or sequentially.

The invention may be practiced in any subject diagnosed with, or at risk of developing, inflammatory bowel disease. Preferably, the subject is a mammal and more preferably, a human.

### Binding of QD Conjugates to Biomarkers

One method of measuring the concentration of a biomarker in a sample is to conjugate QDs to the biomarker and then to detect and quantify the presence of the QDs by fluorescence. The conjugation of QDs to a biomarker can be done by conjugating a QD to an intermediary, such as a targeting moiety, which is selected based on its ability to specifically bind to a biomarker of interest.

A QD conjugate comprises at least one quantum dot (i.e., a semiconductor nanocrystal) that can be detected by means of its fluorescent properties. Quantum dots are ultra-sensitive non-isotopic reporters of biomolecules in vitro and in vivo. QDs are attractive fluorescent tags for biological molecules due to their large quantum yield and photostability. As such, QDs overcome many of the limitations inherent to the organic dyes used as conventional fluorophores. QDs range from 2 nm to 10 nm in diameter, contain approximately 500-1000 atoms of materials such as cadmium and selenium, and fluoresce with a broad absorption spectrum and a narrow emission spectrum.

A water-soluble luminescent QD, which comprises a core, a cap and a hydrophilic attachment group is well known in the art and commercially available (e.g. Quantum Dot Corp. Hayward, Calif.; Invitrogen, Carlsbad, Calif.; U.S. Pat. No. 7,192,785; U.S. Pat. No. 6,815,064). The core comprises a nanoparticle-sized semiconductor. While any core of the IIB VIB, IIIB VB or IVB-IVB semiconductors can be used, the core must be such that, upon combination with a cap, a luminescence results.

The cap or shell is a semiconductor that differs from the semiconductor of the core and binds to the core, thereby forming a surface layer on the core. The cap must be such that, upon combination with a given semiconductor core, a luminescence results. Two of the most widely used commercial QDs come with a core of CdSe or CdTe with a shell of ZnS and emissions ranging from 405 nm to 805 nm.

The attachment group, refers to any organic group that can be attached, such as by any stable physical or chemical association, to the surface of the cap of the QD. In one embodiment, the attachment group can render the QD water-soluble without rendering the QD no longer luminescent. Accordingly, the attachment group comprises a hydrophilic moiety. In one aspect, the attachment group may be attached to the cap by covalent bonding and is attached to the cap in such a manner that the hydrophilic moiety is exposed. Suitable hydrophilic attachment groups include, for example, a carboxylic acid or salt thereof, a sulfonic acid or salt thereof, a sulfamic acid or salt thereof, an amino substituent, a quaternary ammonium salt, and a hydroxy. In another aspect, QD may be rendered water soluble by capping the shell with a polymer layer that contains a hydrophobic segment facing inside towards the shell and a hydrophilic segment facing outside. The hydrophilic layer can be modified to include functional groups such as —COOH and —NH₂ groups for further conjugation to proteins and antibodies or oligonucleotides as described in Chan and Nie, 1998, (Science 281:2016-8), Igor et al., 2005, (Nature Materials 4:435-46), Alivisatos et al., 2005, (Anna. Rev. Biomed. Eng. 7:55-76) and Jaiswal et al., 2003, (Nature Biotech. 21:47-51) and incorporated herein in their entirety by reference.

A QD can be conjugated to a targeting moiety. The targeting moiety specifically binds to the biomarker of interest and may comprise an antibody, a peptidomimetic, a polypeptide or aptamer, a nucleic acid or any other molecule provided it binds specifically to a biomarker of interest. When the targeting moiety comprises an antibody, the antibody preferably specifically binds to a biomarker that is dysregulated during the onset and progression of inflammatory disease. In one embodiment, the antibody specifically binds to a biomarker that is dysregulated by the onset and progression of inflammatory bowel disease. In another embodiment, the antibody specifically binds to a biomarker that is dysregulated by the onset and progression of ulcerative colitis. In still another embodiment, the antibody specifically binds to a biomarker that is dysregulated during the onset and progression of Crohn's disease. Biomarkers of interest in the present invention include, but are not limited to, MPO, or cytokines involved in inflammation, such as IL-1α or TNFα.

In another embodiment, the QD may be conjugated to a targeting moiety comprising a nucleic acid binding moiety. The nucleic acid binding moiety may comprise any nucleic acid, protein, or peptide that binds to nucleic acids, such as a DNA binding protein. A preferred nucleic acid is a single-stranded oligonucleotide comprising a stem and loop structure and the hydrophilic attachment group is attached to one end of the single-stranded oligonucleotide.

The antibody or nucleic acid can be attached to the QD, such as by any stable physical or chemical association, directly or indirectly by any suitable means. Quantum dot conjugation may be achieved by a variety of strategies that include but are not limited to passive adsorption, multivalent chelates or classic covalent bond formation described in Jaiswal et al., 2003 (Nature Biotechnol. 21:47-51) and incorporated by reference herein.

The covalent bond formation is the simplest in execution and hence widely used for conjugation. The antibody or nucleic acid is attached to the attachment group directly or indirectly through one or more covalent bonds. If the antibody is attached indirectly, the attachment preferably is by means of a “linker,” i.e., any suitable means that can be used to link the antibody or nucleic acid to the attachment group of the water-soluble QD. The linker should not render the water-soluble QD water-insoluble and should not adversely affect the luminescence of the QD. Also, the linker should not adversely affect the function of the attached antibody or nucleic acid. If the conjugate is to be used in vivo, desirably the linker is biologically compatible. Crosslinkers, e.g. intermediate crosslinkers, can be used to attach an antibody to the attachment group of the QD. Ethyl-3-(dimethylaminopropyl) carbodiimide (EDAC) is an example of an intermediate crosslinker. Other examples of intermediate crosslinkers for use in the present invention are known in the art. See, e.g., Bioconjugate Techniques (Academic Press, New York, (1996)).

In one embodiment, amine groups on QDs are treated with a malemide group containing a crosslinker molecule. These “activated” QDs can be then be directly conjugated to a whole antibody molecule. However the direct conjugation may result in steric hindrance restricting access of the antibody to the antigen of interest. In those instances where a short linker could cause stork hindrance problems or otherwise affect the functioning of the targeting moiety, the length of the linker can be increased, e.g., by the addition of from about a 10 to about a 20 atom spacer, using procedures well-known in the art. One possible linker is activated polyethylene glycol, which is hydrophilic and is widely used in preparing labeled oligonucleotides.

The Stretptavidin Biotin reaction provides another conjugation method where the biotinylated protein/biomolecule is attached to a streptavidin coated QD.

One of skill in the art will appreciate that it may be desirable to detect more than one antigen or protein of interest in a biological sample. Therefore, in particular embodiments, at least two antibodies directed to two distinct antigens or proteins are used. Where more than one antibody is used, these antibodies may be added to a single sample sequentially as individual antibody reagents or simultaneously as an antibody cocktail. Alternatively, each individual antibody may be added to a separate sample from the same source, and the resulting data pooled.

Quantum dots are conjugated to antibody fragments using a heterobiofunctional crosslinker 4-(maleimidomethyl)-1-cyclohexanecarboxylic acid N-hydroxysuccinimide ester (SMCC). The commercial Quantum dots (Invitrogen Corporation, Carlsbad, Calif.) come with —NH₂ groups on their surface. These amino groups are reacted with the crosslinker SMCC to create malemide groups on the QDs surface. Antibodies of interest are reduced by DTT (Dithiothreitol) and disulfide bonds are broken to create thiol (—SH) groups. The final conjugation relied on the covalent bond formed between the malemide group on activated QDs and the thiol group on the antibodies. The ratio of antibody conjugated to QDs is 1:4 and the typical yield of the reaction at the end of conjugation procedure is anywhere between 500 μl to 800 μl.

Table I presents a list of QDs conjugated to antibodies using the procedure outlined above:

TABLE 1
Different color QDs conjugated to various antibodies.

| | | Stock | |
|---|---|---|---|
| Quantum Dots | Antibodies | Concentration | |
| QD565 | MPO (Santa Cruz BT) | 1.2 | μM |
| QD655 | MPO (Santa Cruz BT) | 500 | nM |
| QD655 | Anti- Testosterone | 1.5 | μM |
| QD605 | Anti-TNFα | 1 | μM |
| QD705 | Anti-TNFα | 1.2 | μM |
| QD 605 | Anti-IL-1α | 1.5 | μM |
| QD 705 | Anti-IL-1α | 1.5 | μM |

### Detection Using QD as Fluorophores

Given the disclosure set forth herein, the skilled artisan will understand how to use any methods available in the art for identification or detection of a protein, nucleic acid, or a biomolecule of interest. Methods for detecting a molecule of interest comprise any method that determines the quantity or the presence of the biomarker protein or nucleic acid.

In one embodiment, the biomarker of interest is detected at the protein level. The method comprises contacting the sample with a QD-antibody conjugate, wherein the antibody of the conjugate specifically binds to the biomarker protein, and detecting fluorescence, wherein the detection of fluorescence indicates that the conjugate bound to a protein in the sample.

In another embodiment, the target molecule of interest is detected at the nucleic acid level. The method comprises contacting the sample with a QD-conjugate, wherein the targeting moiety of the conjugate specifically binds to the nucleic acid, and detecting residual fluorescence, wherein the detection of fluorescence indicates that the conjugate bound to the nucleic acid in the sample. Preferably, the targeting moiety of the conjugate is a nucleic acid. Alternatively, the targeting moiety of the conjugate is a protein or a fragment thereof that binds to a nucleic acid, such as a DNA binding protein.

The term “probe” refers to any molecule that is capable of selectively binding to a specifically intended target molecule, for example, a nucleotide transcript or a protein encoded by or corresponding to a target molecule. Probes can be synthesized by one of skill in the art, or derived from appropriate biological preparations. As contemplated in the present invention, a probe may be conjugated to a QD of a particular size. Examples of molecules that can be used as probes include, but are not limited to, RNA, DNA, proteins, antibodies, and organic molecules.

The present invention also provides a method whereby two or more different target molecules and/or two or more regions on a given target molecule can be simultaneously detected in a sample. The method involves using a set of QD conjugates, wherein each of the conjugates in the set has a differently sized QD or a QD of different composition attached to a targeting moiety that specifically binds to a different target molecule or a different region on a given target molecule in the sample. In an embodiment, the QD of the conjugates range in size from 2 nm to 6.5 nm, which sizes allow the emission of luminescence in the range of blue to red. The QD size that corresponds to a particular color emission is well-known in the art. Within this size range, any size variation of QD can be used as long as the differently sized QD can be excited at a single wavelength and differences in the luminescence between the differently sized QD can be detected. In another embodiment, the differently sized QD have a capping layer that has a narrow and symmetric emission peak. Similarly, QD of different composition or configuration will vary with respect to particular color emission. Any variation of composition between QD can be used as long as the QD differing in composition can be excited at a single wavelength and differences in the luminescence between the QD of different composition can be detected. Detection of the different biomarkers in the sample arises from the emission of multicolored luminescence generated by the QD differing in composition or the differently sized QD of which the set of conjugates is comprised. This method also enables different functional domains of one or more single proteins, for example, to be distinguished.

Accordingly, the present invention provides a method of simultaneously detecting two or more different biomarkers and/or two or more regions of a given biomarker in a sample. The method comprises contacting the sample with two or more conjugates of a water-soluble QD and an antibody, wherein each of the two or more conjugates comprises a QD of a different size or composition and an antibody that specifically binds to a different molecule or a different region of a given target molecule in the sample. The method further comprises detecting luminescence, wherein the detection of luminescence of a given color is indicative of a conjugate binding to a molecule in the sample.

### Diagnostic Assays

The present invention has application in various diagnostic assays for the detection of any inflammatory disease, including, but not limited to IBD, UC, and CD. The present invention can be used to detect inflammatory disease such as IBD by removing a sample to be tested from a patient; contacting the sample with a water-soluble QD conjugated to a targeting moiety that specifically binds to a biomarker associated with a given disease state, and detecting the luminescence, wherein the detection of luminescence indicates the existence of a given disease state, such as IBD. In these eases, the sample can be a cell or tissue biopsy or a bodily fluid, such as blood, serum, urine, or fecal sample.

The biomarker can be a protein, a nucleic acid or enzyme associated with a given disease, the detection of which indicates the existence of a given disease state. The detection of a disease state can be either quantitative, as in the detection of an over- or under-production of a protein, or qualitative, as in the detection of a non-wild-type (mutated or truncated) form of the protein. In regard to quantitative measurements, preferably the luminescence of the QD conjugate is compared to a suitable set of standards. A suitable set of standards comprises, for example, the QD conjugate of the present invention in contact with various, predetermined concentrations of the biomarker being detected. One of ordinary skill in the art will appreciate that an estimate of, for example, amount of protein in a sample, can be determined by comparison of the luminescence of the sample and the luminescence of the appropriate standards, as described in detail elsewhere herein,

### Test Apparatus

An apparatus is provided for practicing one or more Quantum-Linked ImmunoSorbent Assay (QLISA) methods of using quantum dots for detecting antigens indicative of IBD, UC, CD, or other inflammatory disease. The apparatus can be provided in the form of a kit for use in a physician's office or equipment for a diagnostic laboratory. The apparatus includes any manufacture (e.g., a package or a container) comprising at least one reagent, (e.g., an antibody, a nucleic acid probe, etc.) for specifically detecting the expression of a biomarker for IBD, UC, CD, or other inflammatory disease.

The QLISA technology utilizes antibodies conjugated to fluorescent nanoparticles (quantum dots) for detection and quantitation of the desired antigen or antibody, rather than horseradish peroxidase mediated, chemiluminescence based enzyme linked immunosorbent assay (ELISA). The volume of sample required for detecting MPO at picomolar concentrations is thus reduced from 50 μL (96 well plate ELISA set up) to in the range of about 1 μL to about 54. The antibody for capturing MPO is covalently bound to the substrate, as opposed to non-specific binding methods used in traditional ELISA or other immunoassay techniques. Experimental apparatus has been proven to be capable of detecting MPO at picomolar concentrations in solution and in animal samples.

In one embodiment, the apparatus comprises at least two reagents, e.g., antibodies, for specifically detecting the expression of at least two distinct biomarkers. Each antibody may be provided in the apparatus as an individual reagent or, alternatively, as an antibody cocktail comprising all of the antibodies directed to the different biomarkers of interest. Furthermore, any or all of the reagents may be provided within containers that protect them from the external environment, such as in sealed containers.

Positive and/or negative controls may be included in the apparatus to validate the activity and correct usage of reagents employed in accordance with the invention. Controls may include samples, such as tissue sections, cells fixed on glass slides, etc., known to be either positive or negative for the presence of the biomarker of interest. The design and use of controls is standard and well within the routine capabilities of those of ordinary skill in the art.

One of skill in the art will further appreciate that any or all steps in the methods of the invention could be implemented by personnel or, alternatively, performed in an automated fashion. Thus, the steps of body sample preparation, sample staining, and detection of biomarker expression may be automated.

In one embodiment, an apparatus or kit is provided to measure myeloperoxidase (MPO), interleukin 1α (IL-1α), tumor necrosis factor (TNF-α), calprotectin, lactoferrin, fibronectin, anti-sacchharomyces cerevisiae (ASCA), perinuclear anti-neutrophil cytoplasmic antibodies (pANCA) in the stools using a noninvasive measurement technique that can provide robust, sensitive, and specific early detection of inflammatory bowel disease. The apparatus can measure inflammatory biomarkers in biological fluids using QD immobilization and fluorescent light detection.

According to one embodiment depicted schematically in FIG. 1, the apparatus comprises a capillary tube for holding a sample to be analyzed; a needle connected to the capillary tube to provide a sample thereto; an LED or equivalent light source, without or without a focusing lens, to provide an excitation energy to QD conjugates bound to antigens in the sample; and a detection system including an optical detector and a broadband filter to improve signal-to-noise ratio. In one embodiment, the light source is an ultraviolet LED. Alternatively, a laser diode, or a plurality of laser diodes, can be used in place of an LED. In another embodiment, the light source is a violet laser. In yet another embodiment, the light source is a blue laser.

Capillary based assays can present several advantages over conventional 96 well plate methods, including the need for a small amount of analytes and proportionally less volume of required reagents. The confluence of developments in nano-fluidic handling systems has enabled capillary based microreactors and sensors to be employed in high throughput environments. However, the cylindrical nature of the capillary geometry does pose several challenges in the ability to properly couple and collect light (for colorimetric or fluorometric assays), thus limiting the final sensitivity of capillary based assay detection. Some of these challenges can be addressed by more powerful and sensitive optics, and the prior art has approached these challenges by using very expensive customized optical systems and/or electrochemical instruments to enable capillary assays to achieve sensitivity of femtomolar detection. An advance disclosed herein is the design and implementation of an inexpensive, capillary based assay able to detect myeloperoxidase (MPO) at 100 pM sensitivity and at a total volume of about 1 μL and no more than about 5 μl.

Free standing capillary tubes offer superior simplicity in manufacturing and handling compared to developing a full scale lab-on-a-chip type device. Capillary based methods have already been used as immunosensors for detecting trace amounts of explosives, as high throughput automated genome analysis systems, as drug assays, for example in measuring paclitaxel in blood plasma, and even for the detection of helicobacter hepaticus that causes hepatitis in mice. The examples listed above are mostly immunoassays in conjunction with fluorescence spectroscopy. Immunoassays used for the detection of various biomolecules and biochemicals, rely on the interaction between an antigen and its antibody and possess high specificity depending on the antibody-to-antigen interaction. This specificity allows development of assays detecting multiple analytes in one capillary. Enzyme Linked ImmunoSorbent Assay (ELISA) is a commonly followed bioassay that relies on the antigen-antibody specificity and chemistry, with signal amplification capabilities. In a conventional ELISA technique sensing is mostly accomplished by chemiluminescence, although both colorimetric titration or fluorescence can be used.

Fluorescence based ELISA has the capability to detect more than one antigen or antibody by multiplexing. Multiplexing, although a lucrative way to detect multiple markers, has remained thus far a challenge for ELISA methods due to bleed through in the emission bands, the requirement of multiple excitation and emission filter pairs, the low fluorescence life time of fluorophores, and the need for high power light sources. In sophisticated flow cytometry systems (FACS) such bleed through has been accounted for by software but this necessitates complicated data analysis and expensive instruments. Recent developments in quantum dots (QDs) enable significant reduction in photo bleaching due to the unique optical properties of these semiconductor nanocrystals. These properties include single excitation maxima irrespective of emission maxima, and narrow emission spectra which allows multiplexing without any bleed through. Quantum dots (QDs) have found significant applications in biology especially in live cell imaging to follow and understand signaling pathways. Although commercially available QDs are expensive on a per pound basis, the ability to carry out reactions in nano to microliter volumes coupled with moderately sensitive CCD cameras or photon counters can produce a cost-effective assay; the raw material costs per unit mass remain low due to the small amount of QDs necessary to carry out the assay.

Both glass and polymer based capillaries have been used to carry out immunoassays. Specifically, fused silica, polystyrene, polymethylpentene, and polymethyl methacrylate have been used in fabricating capillary biosensors to estimate biomarkers in a volume range of 0.5 to 5 μL. Any transparent polymer material capable of transmitting light down to 365 nm, including polycarbonate, can be used for making the capillary tubes. Polymeric capillaries are of particular interest due to readily available functional groups on their surface offering an appropriate substrate for immobilizing antibodies or antigens. Furthermore, photochemical methods are available to functionalize polymeric materials. Several strategies have been devised to detect low concentrations of antigens by solid phase immunoassay in capillaries, primarily focusing on excitation of fluorophores followed by collection of the emitted photons. One such approach takes advantage of the evanescent field at the interface of the polymer/liquid interface for collecting the emitted signal; this particular method requires the material of the capillary to function as a waveguide. The excellent optical properties of PMMA allow use of PMMA capillaries as waveguides and fabrication of sensors to measure optical properties of molecules.

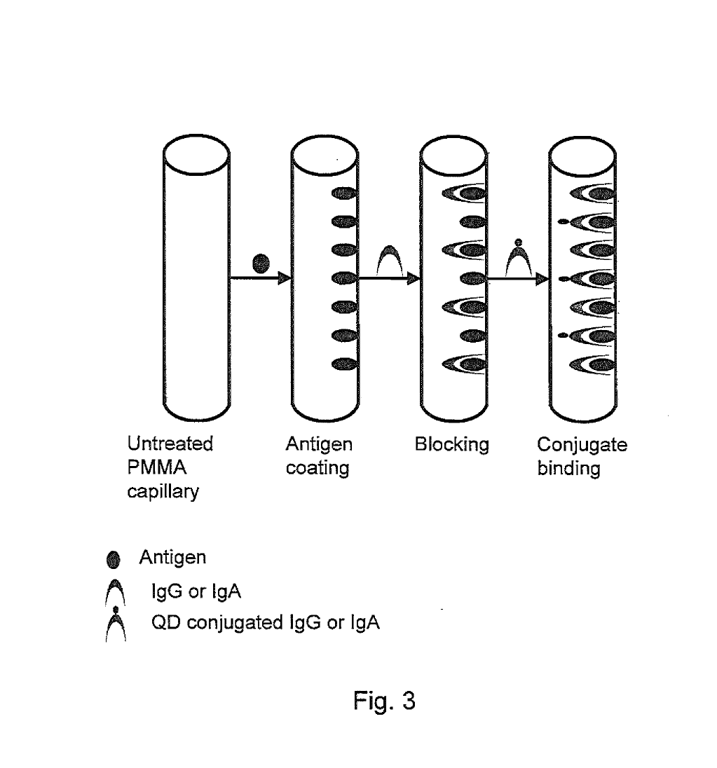

In one QLISA method using an apparatus as disclosed herein, antigen can be captured and analyzed at levels ranging as low as picograms to nanograms. With reference to FIG. 3, in one embodiment, the method comprises providing an untreated polymethyl methacrylate (PMMA) capillary tube, coating the capillary tube with antigen, blocking antigens that are not disease specific, and binding disease specific conjugates to the remaining antigens. In another embodiment, the method comprises functionalizing a PMMA capillary tube, conjugating an appropriate polyclonal antibody specific to the antigen desired to be measured within the capillary tube, reacting the antibody-antigen complex with a quantum dot tagged secondary antibody specific to the antigen, exposing the capillary tube to a light source to excite the quantum dots, and determining the concentration of the antigen by measuring the fluorescence of the quantum dots. In particular, an untreated PMMA tube is coated with antigen, nonspecific antigens are blocked with antibodies such as immunoglobulin, as is well known in the art, and specific antigens are bound to quantum dot conjugated antibodies. As depicted schematically in various configurations in FIGS. 6A-9, 11A-11D, and 16A-16B, excitation photons can be provided directly through one or both ends or through the sidewall of the capillary tube, and emitted photons can be collected into a spectrometer or CCD camera either directly or via a fiber optic cable. Alternatively, another optical emission detectors, including but not limited to photomultiplier tubes (PMTS), avalanche photodiode detectors (APDs), multi-pixel photon counters (MPPCc) can be used.

The apparatus includes a low cost PMMA microcapillary biosensor using QDs as the fluorescent probe for detection of picomolar quantities of analytes. PMMA is a preferred capillary material because of its optical properties and the capability to selectively functionalize its surface for antibody immobilization. Capillary dimensions of 250 μm ID and 2.5 cm long allow for a volume of about 1 μl. Such capillaries are commercially available. The high quantum yield of QDs coupled with the ability to excite QDs that emit at different wavelengths with a single UV light guided the choice of reporter probes. The inexpensive capillary based immunofluorescent assay described herein was used for detecting and estimating the concentration of myeloperoxidase, an inflammatory marker over-expressed in inflammatory diseases including those of the gastrointestinal tract.

The use of a PMMA microcapillary biosensor in combination with QDs as the fluorescent probe for detection of picomolar quantities of analytes has been demonstrated to be effective at detecting myeloperoxidase (MPO). The selection of PMMA was based on its optical properties and the capability to selectively functionalize its surface for antibody immobilization. Capillary dimensions of 250 μm ID and 2.5 cm long allow use of a volume of ˜1 μl, and these capillaries are commercially available. The high quantum yield of QDs coupled with the ability to excite QDs that emit at different wavelengths with a single UV light guided the choice of reporter probes. The inexpensive capillary based immunofluorescent assay described herein has proved useful for detecting and estimating the concentration of myeloperoxidase, an inflammatory marker over-expressed in inflammatory diseases including those of the gastrointestinal tract. Thus, a low cost, robust immunofluorescence sensor has been developed, the sensor being capable of operating with 1 to 2 μL of analyte and detecting subnanomolar concentrations. The capillary immunoassay methodology and design discussed herein can be further improved with regard to sensitivity, but for many clinical applications the sensitivity demonstrated by herein is adequate to distinguish diseased from healthy individuals. Improvements to increase sensitivity are possible both by chemistry optimization approaches as well as with more elaborate optics. However, a focus of the present disclosure is on a low cost easy to deploy assay that is a substantial advance over anything in the prior art.

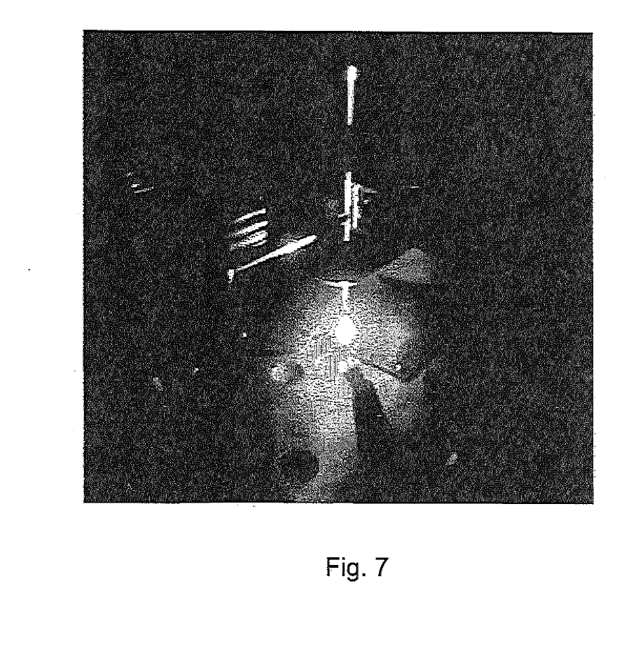

As depicted in FIGS. 6A and 6B a test apparatus includes a capillary tube for containing a sample tagged with QD conjugates, an LED light source to excite the quantum dots, a spherical or flat mirror to concentrate the fluorescence emitted by the quantum dots, and an optical detection system for detecting and measuring the fluorescence signal. Alternatively, a cylindrical or parabolic reflector or mirror can be used to concentrate the fluorescent emissions form the quantum dots. The optical detection system can include a broadband filter to improve signal quality, and can utilize a photodiode based detector, a spectrometer with one or more photomultiplier tubes, a CCD camera, or other optical detection system, depending on the sensitivity required. In one embodiment, QD intensity is be measured using a standard fluorescence meter (Fluoromax 3), which permits determination of QD bioconjugates concentrations of the order of femto molars. A photograph of an apparatus in operation is shown in FIG. 7.

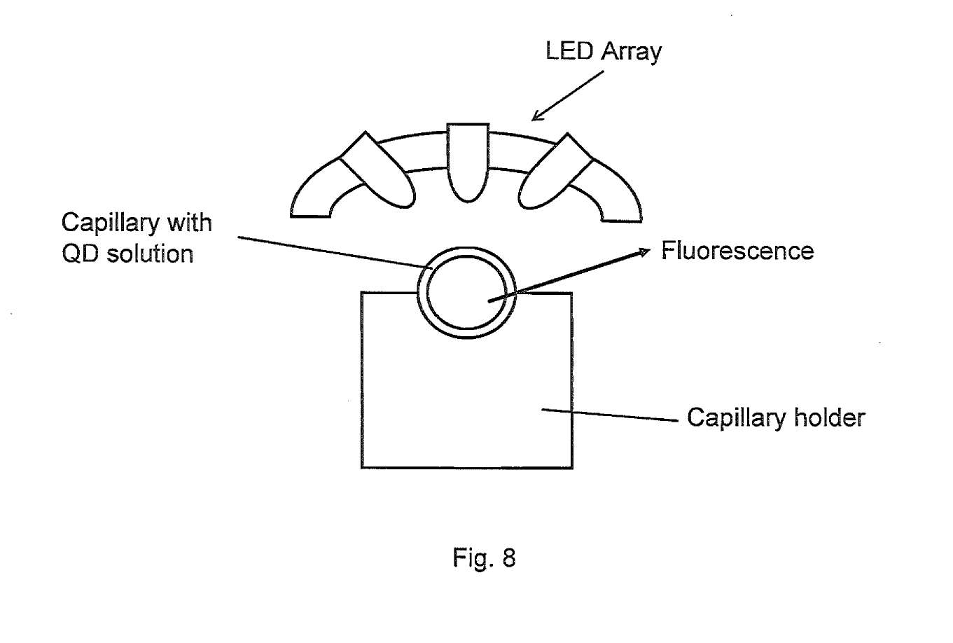

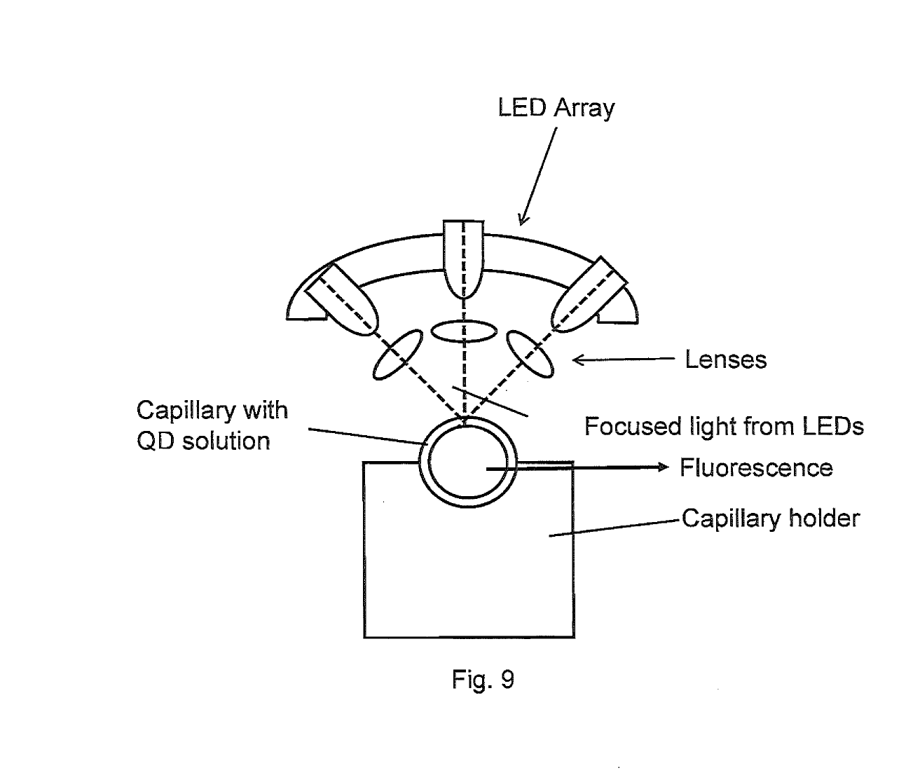

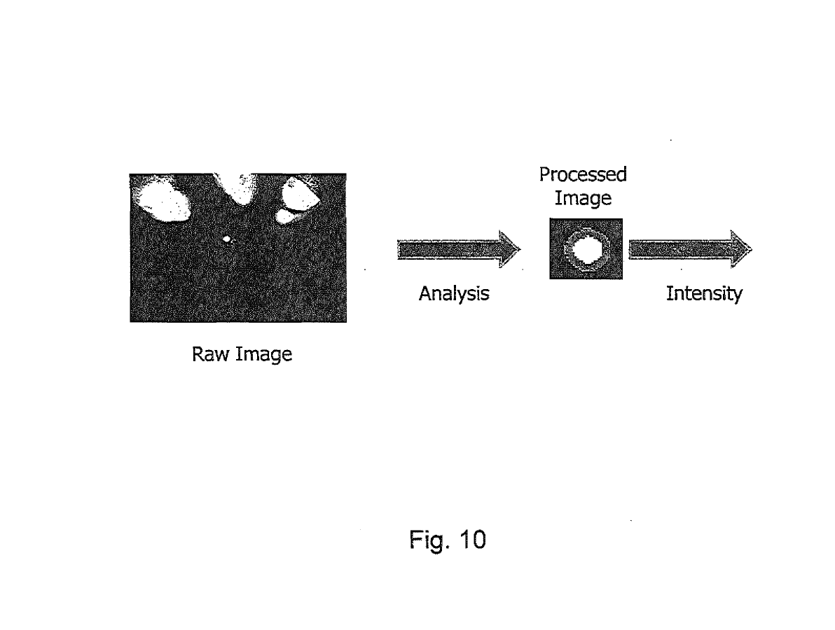

FIG. 8 depicts a light source comprising a plurality of LEDs arranged around a capillary tube, and FIG. 9 depicts a light source comprising a plurality of LEDs focused by lenses arranged around a capillary tube. By using multiple LEDs, with or without lenses, more power can be supplied per unit volume of the capillary tube to increase the fluorescence intensity emitted by the quantum dots. In an exemplary embodiment of the apparatus as depicted schematically in FIGS. 8 and 9, the entire apparatus has dimensions of about 1 inch square. FIG. 10 shows a raw image obtained by a configuration as in FIG. 8, and a processed image from which the QD fluorescence intensity can be determined.

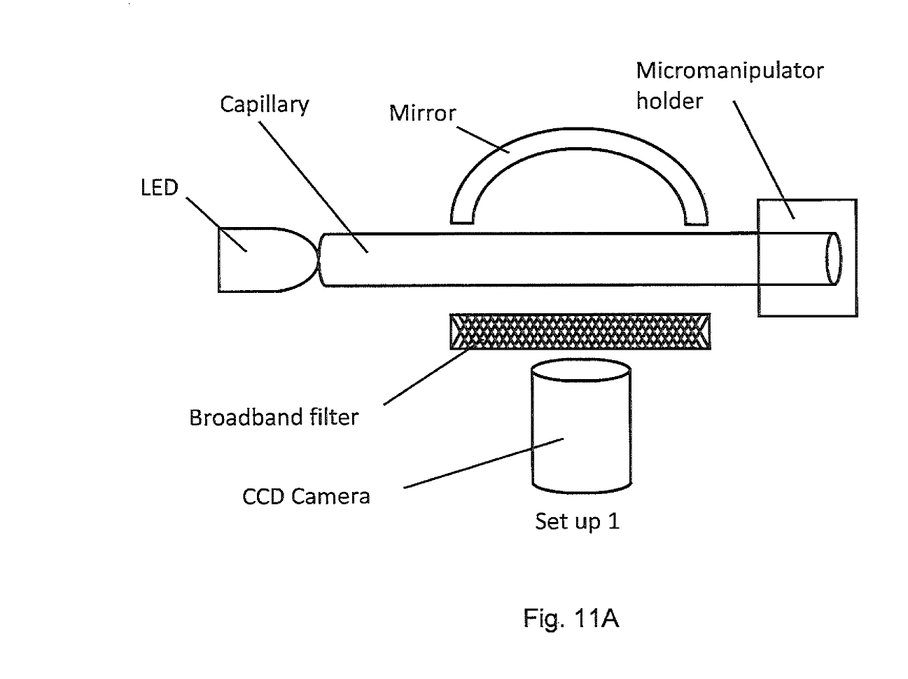

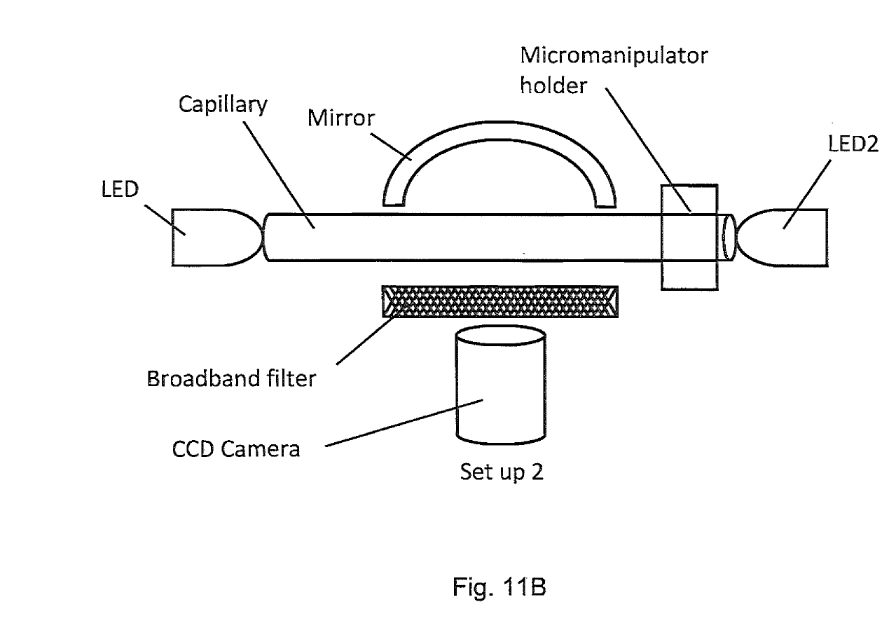

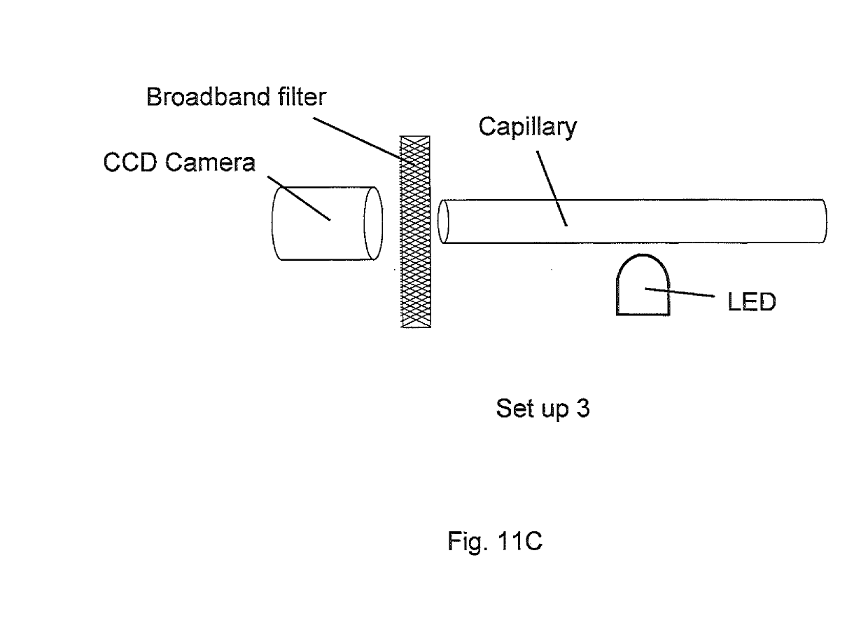

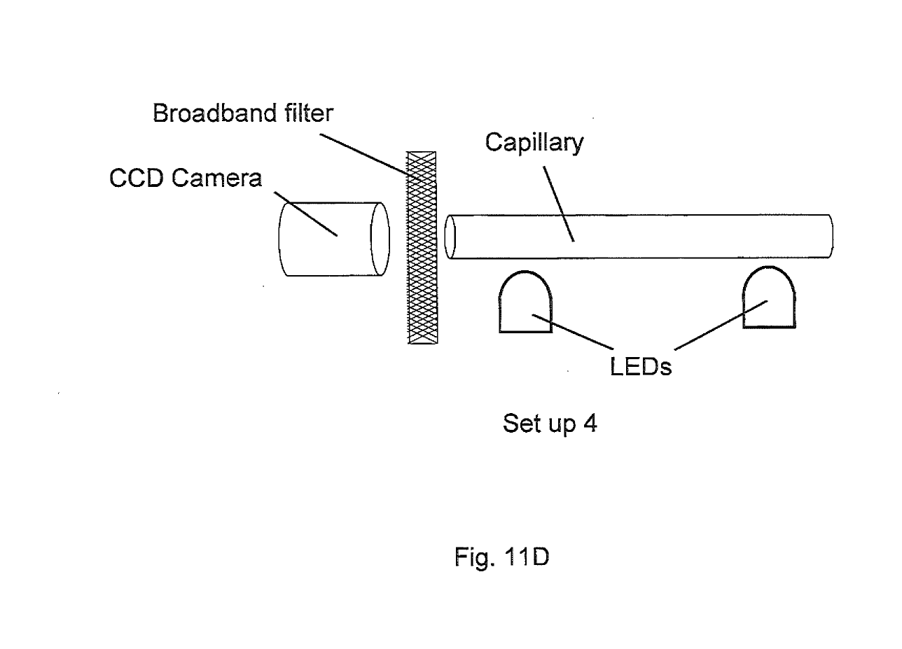

FIGS. 11A-11D depict various arrangements of an apparatus for carrying out the QLISA analytic method. In FIG. 11A, a capillary tube is held in place by a micromanipulator at one end of the capillary tube and an LED light source is provided at an opposite end of the tube. A spherical mirror is provided around at least a portion of the tube to concentrate the fluorescent emissions of the quantum dots to a CCD-based optical detection system. In FIG. 11B, a micromanipulator is arranged to be away from the ends of the tube so that a first LED can be provided at one end of the tube and a second LED can be provided at an opposite end of the tube, to enhance the excitation energy and thus the fluorescent emission of the quantum dots. In FIG. 11C, an LED light source is provided adjacent to a capillary tube to provide excitation energy to the quantum dots, and a CCD-based detection system measures fluorescent emissions from one end of the tube. In FIG. 11D, the arrangement of FIG. 11C is enhanced by the addition of at least one more LED light source. In one embodiment, the apparatus configuration uses one capillary per measurement.

### QLISA Method

An exemplary QLISA method for diagnosing IBD is shown in FIG. 2. A stool sample is provided to the test apparatus. As a threshold test, the presence or absence of MPO and/or calprotectin (and/or lactoferrin) can be detected to determine whether the patient has irritable bowel syndrome (IBS) or inflammatory bowel disease (IBD). For a sample indicating IBD, the amount of MPO and/or calprotectin (and/or lactoferrin) can be quantified to determine the severity of the condition. To further diagnose whether the IBD condition is Crohn's disease (CD) or ulcerative colitis (UC), the apparatus can be used to measure ASCA (indicative of CD) and pANCA (indicative of UC). Although the flow chart of FIG. 2 shows the detection and measurement steps being performed sequentially, the QLISA method can be applied to simultaneously detect the presence and concentration of MPO, calprotectin, lactoferrin, ASCA, pANCA, and any of various other antigens, provided that each antigen is tagged with a different wavelength QD conjugate, as described herein.

The quantity of sample required to detect an antigen using QLISA is in the range of 100 nanoliters to 1.5 microliter, as compared with existing methods such as ELISA, which require 50 to 100 microliters. Experiments with using QLISA have shown that concentrations of antigen as low as 100 picomolar to 10 nanomolar can be detected in animal stool samples of 1 microliter, as compared with concentrations of 1.25 to 62.5 picomolar detectable by ELISA. When starting with a primary immobilized capillary tube, a QLISA analysis can be performed in approximately 3 hours. In one embodiment, a test kit is provided comprising a single assay (MPO and other markers in stool samples for IBD) with customized antibody-coated micro-columns (0.025 μl in volume) and ready-to-use reagents for rapid and easy detection.

An exemplary procedure is described herein for MPO antibody, noting that a substantially identical procedure can be used with respect to other selected biomarkers, including but not limited to calprotectin, lactoferrin, p-ANCA, ASCA.

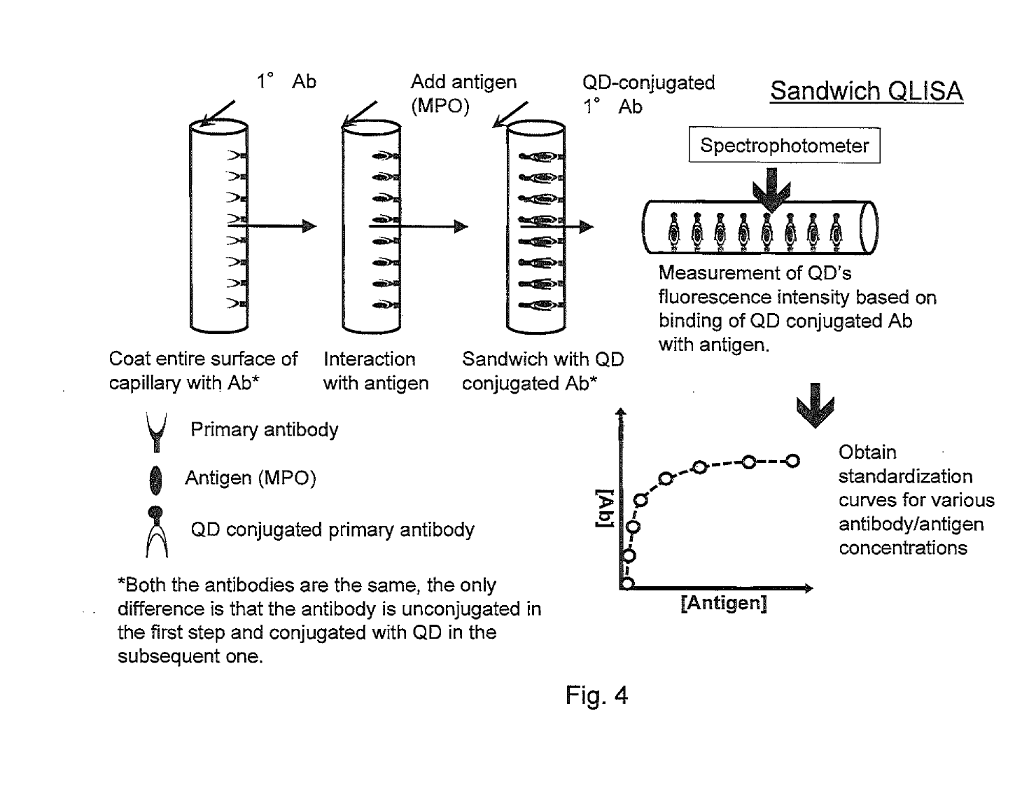

An immobilization assay using QLISA can be performed with the test apparatus or kit to detect nanoscale quantities of desired biomarkers based on a sandwich assay or a competitive assay. In a sandwich QLISA assay, as depicted in FIG. 4, a sample is sandwiched between two antibodies. A monoclonal antibody against MPO is used to sandwich the MPO (antigen) between the primary anti-MPO antibody and QD conjugated anti-MPO monoclonal antibody. The methodology is simple, rapid and catered to a point of care service. An advantage of this method is that the MPO present in the biological sample need not be purified and unconjugated QDs present in the mixture will not bind MPO and will be removed during washing. The assay can be optimized for detecting MPO at femto to picomolar levels, because the detection methods require only minimal binding of MPO. An appropriately functionalized polystyrene (PS), polyvinylacetate (PVA) or polyvinylchloride (PVC) substrate can be used. In one embodiment, a polystyrene capillary is used, the inner wall of the capillary being coated with primary unconjugated monoclonal antibody specific to MPO. A sample potentially containing MPO is injected into the capillary, and the capillary is then washed to remove any excess MPO not attached to the antibody on the column. QD-MPO antibody conjugates are the passed through the column and are allowed to interact with the capillary. Binding occurs between the already immobilized MPO and the QD-MPO antibody conjugates. The capillary is again be washed to remove unconjugated QDs.

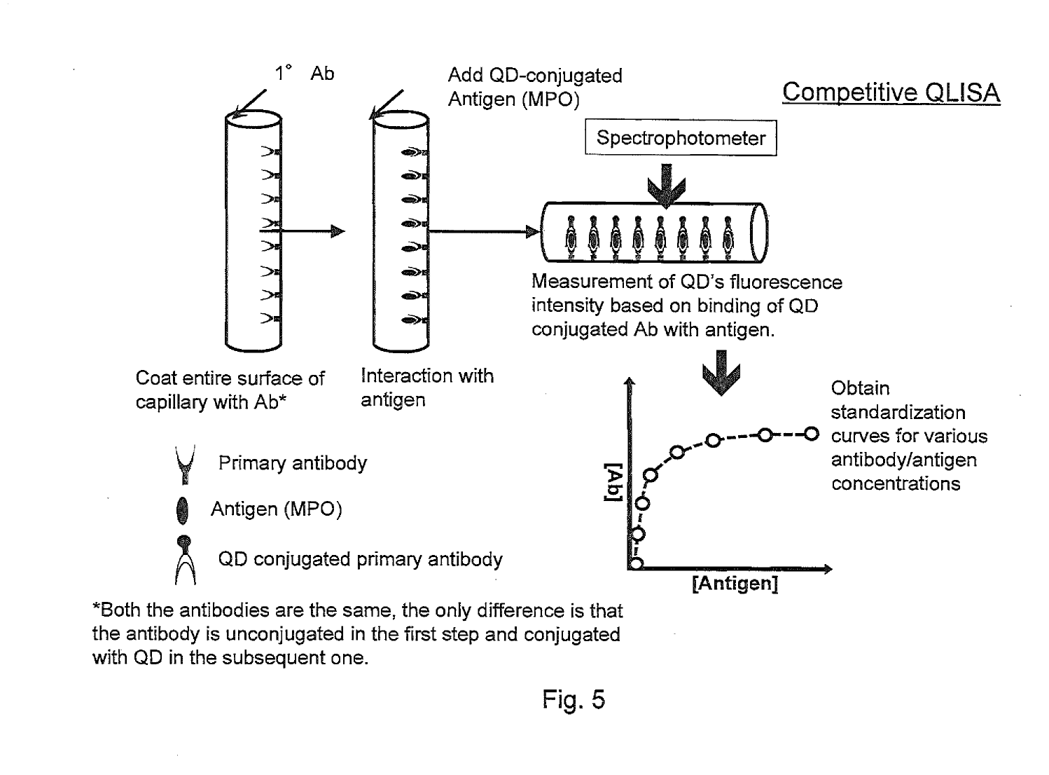

In a competitive QLISA assay, as depicted in FIG. 5, a sample competes with a known antigen for antibody binding. In the competitive assay, the primary antibody is coated on the surface of a polyvinylchloride (PVC) capillary. A sample potentially containing unlabeled MPO is injected into the capillary, the MPO binding to the primary antibody. QD-conjugated MPO is then added to bind the still available primary antibody coated to the capillary tube. The QD-conjugates bind to the primary antibody wherever binding sites are not already occupied by unlabeled MPO. Therefore the more unlabeled MPO present in the sample, the lower the amount of conjugated MPO that binds in the column.

A methodology of surface preparation and immobilization is also provided to take advantage of Quantum Dots by preparing flat PMMA well plates where the functionalization chemistry of the capillary can be carried out. These well plates can be read a conventional ELISA reader. Although the well plates would not necessarily be useful in the needle and capillary apparatus discussed herein, other applications may more easily be converted to use flat substrates.

A person of skill in the art of ELISA measurements and other similar diagnostic techniques will be familiar with sandwich and competitive assay techniques, so further detailed explanation is not deemed necessary. Using a QLISA apparatus as provided herein, QD conjugates can reliably detect and measure antigen expression, which can readily be correlated with disease activity indices. These assays are of value and use to a variety of conditions requiring quantification of biomarkers beyond IDS, including but not limited to transplant rejection (cytokine detection), heart disease (MPO, CRP), and rheumatoid arthritis.

As described herein, quantum dots can be conjugated to antibodies, including but not limited to MPO, IL-1α, TNFα, lactoferrin, calprotectin, and nonspecific antibodies. Further, colitis disease activity can be correlated to expression of MPO alone, or expression of a combination of MPO, IL-1α, and TNFα. The expression can be measured by fluorescence intensity of QD conjugates and the expression correlated with disease activity index in the DSS model of colitis. In particular, calibration curves can be established for antibody coated micro-capillary tubes.

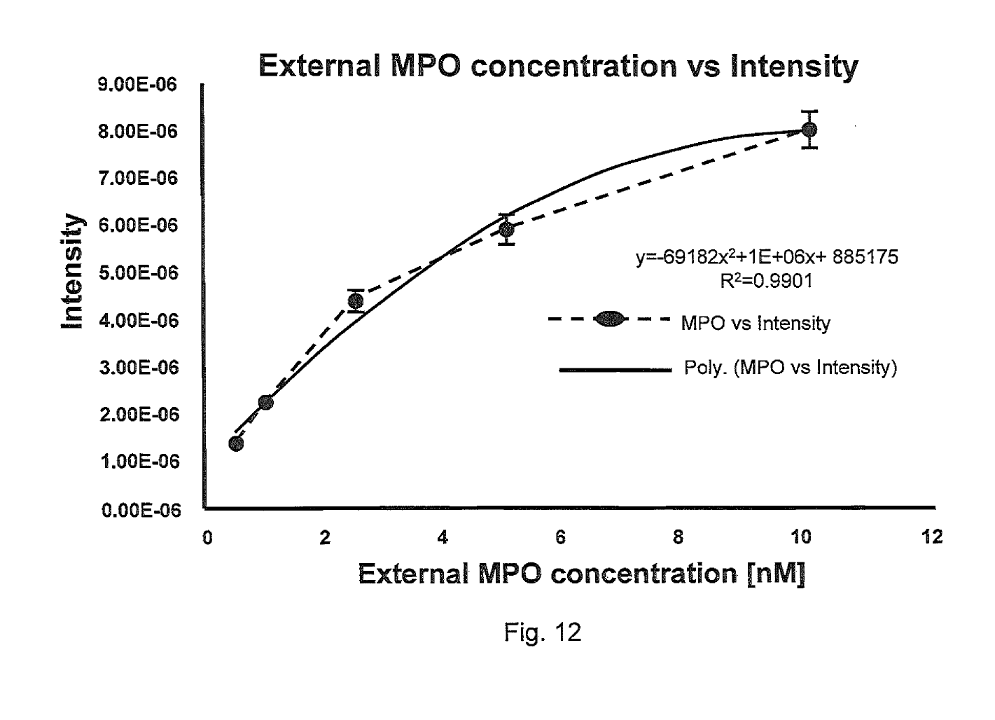

Experimental studies were conducted to quantify the biomarkers for IDS. The biomarkers selected were visualized with the corresponding QD conjugate and their expression was quantified using simple image quantification techniques. The results demonstrate increase in the intensity of biomarkers with increased inflammation. An excellent correlation was established, as depicted in FIG. 12, between the quantified intensity of MPO and disease activity index which is based on clinical parameters. Similar correlations can be established for other biomarkers.

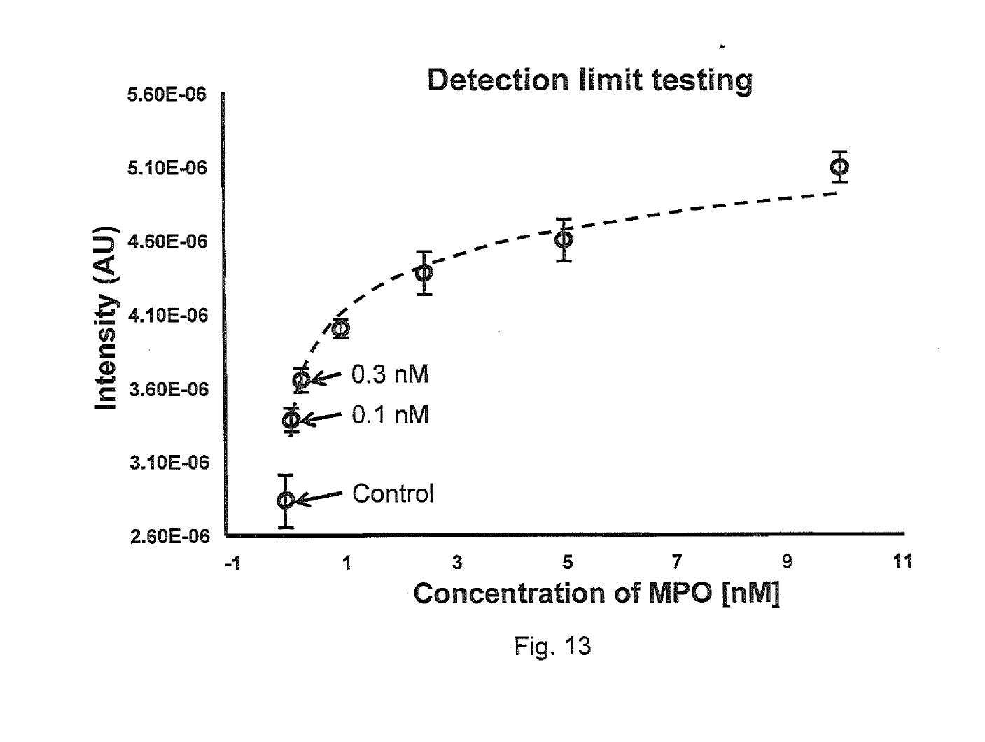

With reference to FIG. 13, MPO has been successfully captured at concentrations as low as 100 picoMolar in solution. Known concentrations of MPO were captured by polyclonal antibody immobilized on the surface of PMMA capillary tubes. Monoclonal antibody against MPO was conjugated to QDs (λₑₘ=605 nm) and allowed to react with the captured MPO. Washing excess antibodies and blocking the surface to prevent nonspecific binding were carried out inside the capillaries using a syringe pump. Capillaries were then imaged using a monochromatic CCD camera and the average intensity as a function of MPO concentration was obtained.

FIG. 13 demonstrates the increase in fluorescence intensity as a function of MPO concentration. The minimum detectable concentration of MPO at a 95% confidence interval was found to be 100 picoMolar, when using an 80 mW/cm² UV LED as the light source. The minimum detectable concentration can be further reduced by taking steps to increase the signal to noise ratio. In particular, it has been determined that the resolution of the QLISA assay in capturing MPO with the current optical system is approximately 10 picoMolar. Improvements in the optical system can further increase the resolution. The greater the resolution, the more effective the QLISA method in situations in which sensitivity is of paramount importance, for example in the early detection of cancer.

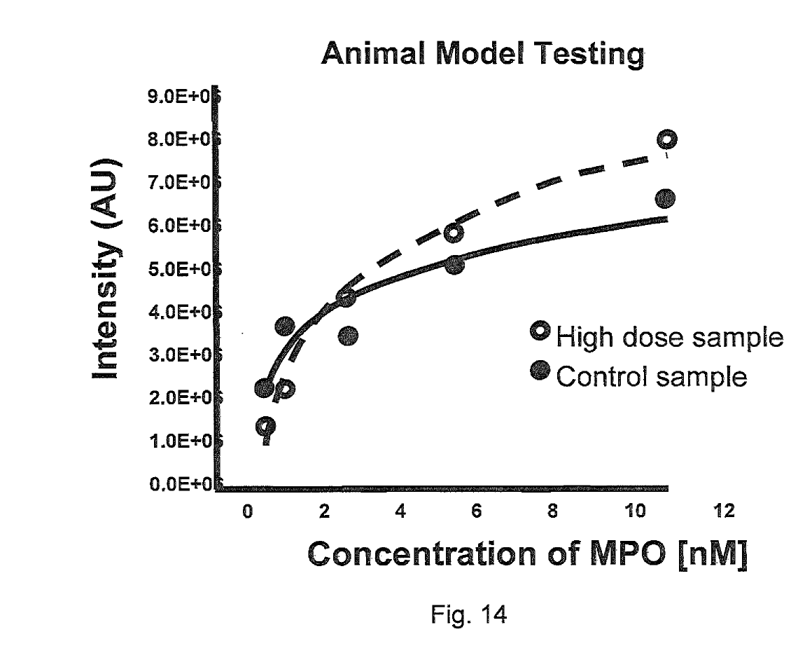

The ability of the QLISA protocol to detect MPO in the presence of non-specific binding to other proteins was demonstrated using animal samples. FIG. 14 shows the concentration of MPO in spiked samples from stools of animals with disease simulating IBD. A statistically significant difference between the control samples and the 10 nm MPO spiked samples indicates that protocol developed can be used in animal samples. Similarly, functionalized quantum dots have been used to quantitatively assess the presence of Myeloperoxidase (MPO), Interleukin 1α (IL-1α), and Tumor Necrosis Factor-alpha (TNF-α), either alone or in combination, in tissues and have correlated biomarker expression to clinical disease activity in the Dextran Sulfate model of colitis.

### Experimental Results

An approach for detecting MPO is a sandwich assay depicted in the flow chart of FIG. 15. (Note that the detection of MPO, as depicted in the flow chart, can be supplemented with detection of lactoferrin.) Briefly, a polyclonal MPO antibody (pAb) is immobilized on the inner surface of a PMMA capillary (capture antibody). MPO from the sample of interest is captured by this pAb and immobilized on the surface. Addition of a QD-mAb complex allows MPO detection by fluorescence.

PMMA capillaries (pCAPs) were selected for use in a sensitive assay due to the readily available functional groups on the surface of PMMA and the excellent optical properties of PMMA. Capillaries used in the experiments disclosed herein were obtained from Paradigm Optics Inc. PMMA capillaries having an outside diameter of 500 microns and an inside diameter of 250 microns. The capillaries were cut into 3 cm long pieces, either before or after functionalization depending on the experiment, and held straight using a custom-built spring loaded holder. The holder eliminated the natural tendency of PMMA capillaries to “buckle.”

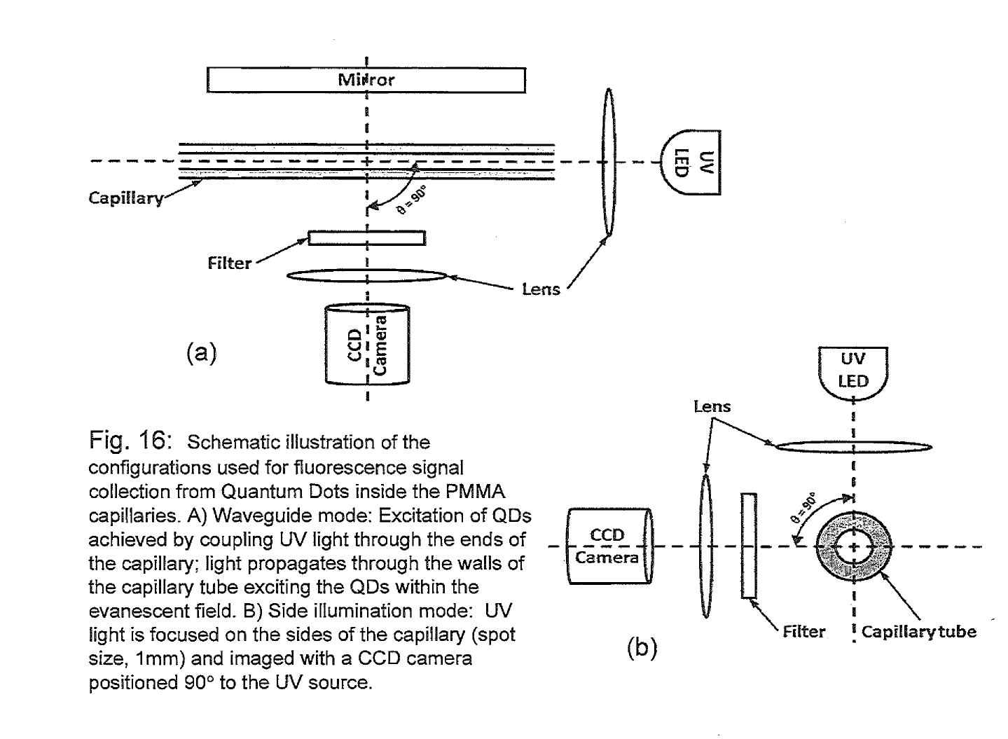

Functionalization of PMMA capillaries was carried out by alkaline ester hydrolysis of the methacrylate on the inner walls of the capillary following a known method. Briefly, 1N NaOH at 60° C. was pumped through the PMMA capillary using a peristaltic pump (100 μL/min) for one hour followed by washing with 1×PBS buffer (pH 7.4). This step hydrolyzes the acrylate ester group on the surface of the PMMA capillary resulting in COOH termination that is crucial for covalently bonding the MPO antibody to the inner walls of the capillary. A rabbit anti-human polyclonal MPO antibody was purchased from ABD Serotec, Raleigh, N.C., USA. Funetionalized PMMA capillaries were then treated with EDC/NHS (104.7 mM EDC 21.7 mM NHS) for 5 hours followed by loading the MPO capture antibody using a concentration of 100 nM. Optimal immobilization of the polyclonal MPO antibody on the inner surface of the PMMA capillary was accomplished by incubation at 4° C. for 16 hours. Non-immobilized antibodies were then removed from the capillary by washing with a buffer containing 0.1% Tween and 0.03% sodium azide (purchased from Sigma Aldrich) in 1×PBS at pH 7.4. Subsequently, a blocking buffer containing 2% FBS in 1×PBS buffer was introduced into the capillaries to reduce nonspecific binding of proteins, and excess blocking buffer was washed away with the same wash buffer. The desired analyte, 1 μL of pure MPO or properly prepared animal sample was then introduced into the pAb immobilized capillaries with the aid of a Hamilton septum adapter and allowed to interact with the pAb for one hour at room temperature followed by injection of wash buffer at a flow rate of 50 μL/min. Monoclonal anti-human Myeloperoxidase antibody (mAb) conjugated to amine terminated QDs (λₑₘ=605 nm, from Invitrogen) was used as the reporter molecule. Conjugation of QDs to mAb was carried out by following the protocol provided by Invitrogen. Both MPO and the mAb were purchased from Lee Biosolutions Inc. QD conjugated mAb (QD-Ab) at 100 nM concentration was then introduced into the PMMA capillary, incubated at room temperature for one hour followed by washing with the wash buffer. The intensity of the QD-Ab from the capillaries was obtained by the optical set up shown in FIG. 16B and described in detail below. Several capillaries were imaged at various steps of the process to evaluate and optimize the immobilization and reaction conditions by fluorescence microscopy (Leica DMRX upright fluorescence microscope). Calibration curves were generated using MPO solutions of known concentrations and this allowed evaluation of the lowest detection limit (LDL), and establishing the sensitivity of the assay. The amount of fluid inside the capillary was restricted to 1-2 μL by limiting the length of the liquid plug inside the capillary. The QLISA assay was then carried out to determine its sensitivity and selectivity for detecting MPO in solution and in animal stool samples. Various steps involved in the QLISA protocol are summarized in FIG. 15.

### Optical Detection and Optimization of Optical Systems

PMMA capillaries (pCAPs) were inserted in a larger glass capillary for experiments where the capillaries acted as waveguides (FIG. 16A), and in those cases a cylindrical mirror was used to collect the scattered fluorescent light. UV-LEDs rated at 10 mW and 80 mW optical power from Nichia Corporation were used as the excitation source and a bandpass filter (600±20 nm) was used on the detection side to remove any UV signal from the excitation source. An aspheric lens (f=6 mm) served the purpose of concentrating the UV light to a spot size of ˜1.5 mm while a separator mounted in front of the lens holder allowed us to position the capillary (irrespective of the experimental configuration) in the focal plane of the lens. A three axis manual positioner was used to align the mirror or the UV source depending on the experiment. Various configurations were tested to assess the capability of the system as shown in FIGS. 16A and 16B, Capillaries containing relatively high concentrations (>1 nM) of MPO or calibration experiments with unconjugated QD solutions were imaged with a monochrome CCD camera (COHU 4900). A firewire monochrome CCD camera (Stingray, AVT-FS-033B) purchased from 1stVision Incorporated was used in all capillaries where the MPO concentration was lower than 1 nM, BMP images (640×480 px) were collected using a frame grabber (VCE-Pro, PCMCIA, Imprex Inc.). Images collected from the CCD were analyzed and resulted in quantification of the concentration of MPO based on the fluorescence intensity of the QD-MPO-Antibody. The average intensity value of pixels within a 30×15 pixel window located between the inner walls of the capillary was obtained using Image.

After optimization of the signal to noise ratio, the lowest detection limit (LDL) of the QLISA protocol in PMMA capillaries was determined by capturing MPO (0.1 to 10 nM) in solution and is referred to as the assay sensitivity of detection. Single low power LED (LPLED) with λₘₐₓ=375 nm was used in a waveguide mode as illustrated in FIG. 16A. The antibody immobilization step was carried out with 100 nM pAb. Use of higher concentrations of pAb did not demonstrate any significant increase in the sensitivity level of MPO detection. The highest concentration of QD-Abs used in all experiments was 100 nM; non-specific binding is expected to be maximum at this concentration irrespective of the blocking step.

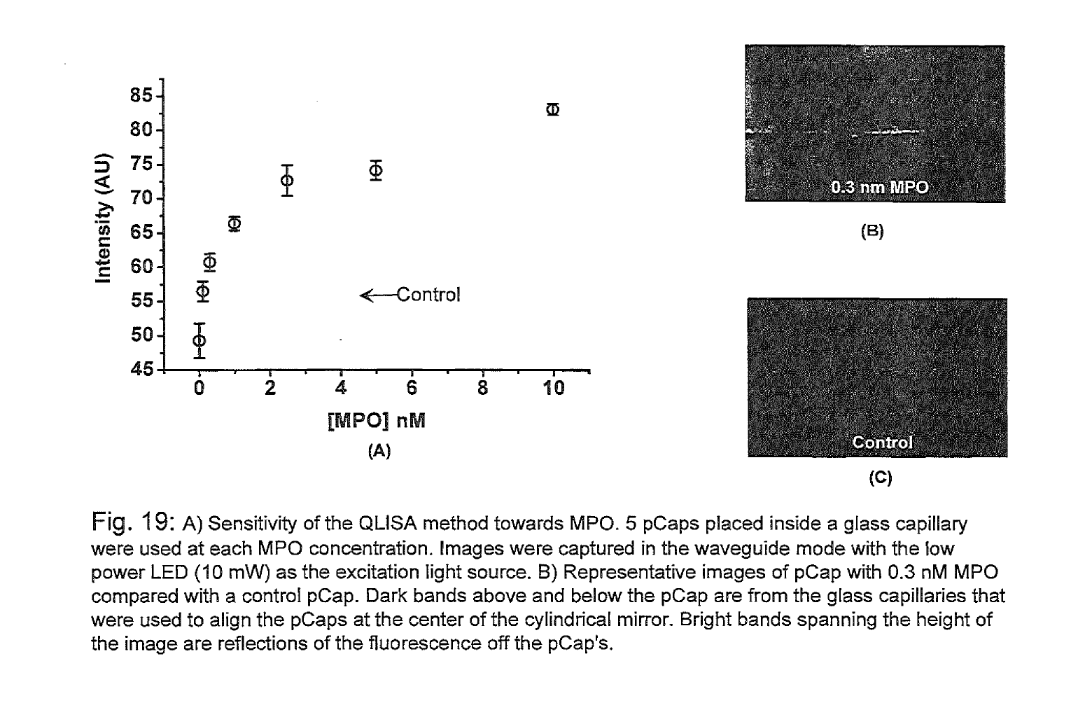

FIG. 19A demonstrates a non-linear relationship between the concentrations of MPO in solution and the fluorescence intensity obtained using the capillary in a waveguide mode with the low power LED (10 mW) as the excitation light source. Five PMMA capillaries were used at each MPO concentration. Detection down to 300 pM of MPO is possible, but the resolution of the system decreases at concentrations below 1 nM. Regardless of the loss in resolution, intensity values obtained at these low concentrations were still above the control at a statistically significant level (p values for t-test are 0.05 and 0.0065 when comparing the control to 100 pM and the control to 300 pM respectively).

Representative CCD images of PMMA capillaries used for detecting 0.3 nM of MPO and a control PMMA capillary are shown in FIG. 19B. An average intensity of QD fluorescence was calculated from image analysis of the bright bands within the capillaries. The dark regions above and below these bright bands represent the UV absorption of the glass capillary; these glass capillaries were used to align the PMMA capillaries in the focal plane of the cylindrical mirror.

The loss in resolution at lower concentrations is believe to primarily be due to limitations in the optical hardware i.e. the CCD camera, an analog camera with maximum integration time (shutter speed) of 16 ms. In addition, at such low concentrations, fluorescence measurements become more sensitive to any inhomogeneities in QD coverage. Furthermore, since PMMA capillaries are not as rigid as glass capillaries, perfect alignment on the focal plane of the mirror and the excitation source becomes exceedingly difficult. To overcome this challenge, we used a larger glass capillary as a support for the PMMA capillary during imaging; PMMA capillaries were left protruding out slightly towards the excitation source in order to minimize interference from the glass capillary. Although this approach works with difficulty in the laboratory, it is recognized that a commercial implementation may require a more robust arrangement to decrease the amount of time required to manually align the capillaries in the optical system. However, rather than trying to optimize the waveguide approach for a commercial set up, a more efficient arrangement for a scalable, cost effective optical immunosensor based on quantum dots is disclosed. In particular, the experimental results indicate that the waveguide approach, which would have been chosen by those skilled in this field based on prior work, is not optimal and under-utilizes the efficiency of quantum dots. Additionally, Low Power LEDs themselves have inherent fluorescence which results in further loss in sensitivity. Therefore, it was determined that a side illumination mode or configuration (FIG. 16B) and high power LED (HPLED) substantially circumvented the problems of using a LPLED in a waveguide mode and improve the sensitivity of the assay. Moreover, the side illumination mode minimized, and likely could be optimized to eliminate the time consuming issues pertaining to optical alignment.

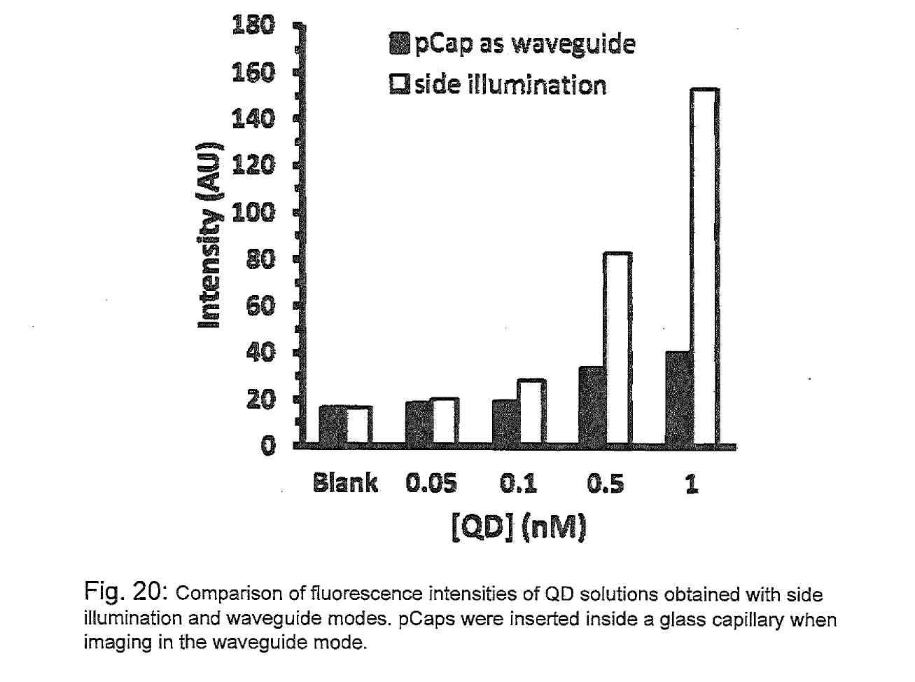

The effect of the mode of excitation i.e. side illumination as compared with waveguide mode, is shown in FIG. 20, using a high power LED (80 mW) as the UV excitation source. PMMA capillaries were loaded with QD solutions of known concentration and imaged in both modes with a total volume of the liquid plug at ˜1 μL. The advantages of side illumination can be clearly seen, although the difference diminishes at concentrations below 100 picoMolar (0.1 nM).

The loss in detection sensitivity by using the PMMA capillary as a waveguide in the disclosed system can be attributed to several factors: 1) optical misalignment in the waveguide mode leading to reduced coupling efficiency, 2) insufficient path length resulting in decreased fluorescence volume, and most importantly 3) availability of only few QDs at the PMMA/solution interface; since the penetration depth of the evanescent field is between 30-300 nm from the surface, only those QDs that are within that range get excited.

In contrast, side illumination results in exciting almost all of the QDs in a volume of ˜50 nL (beam diameter=1 mm), which could be the main reason for the observed difference in intensities between the two illumination modes at relatively higher concentrations (>100 pM). As the concentrations approach the 50 pM range the difference in intensity is practically zero. This suggests that although a capillary tube acting as a waveguide can be the perfect solution for single molecule spectroscopy, side illumination appears to be a better choice due to its simplicity and superior performance at higher concentration levels. These concentrations levels, although higher than single molecules, are still at the nanomolar detection range and can be relevant to biomarkers of human disease.

### Specificity of QLISA Protocol Applied to MPO and Assay Optimization

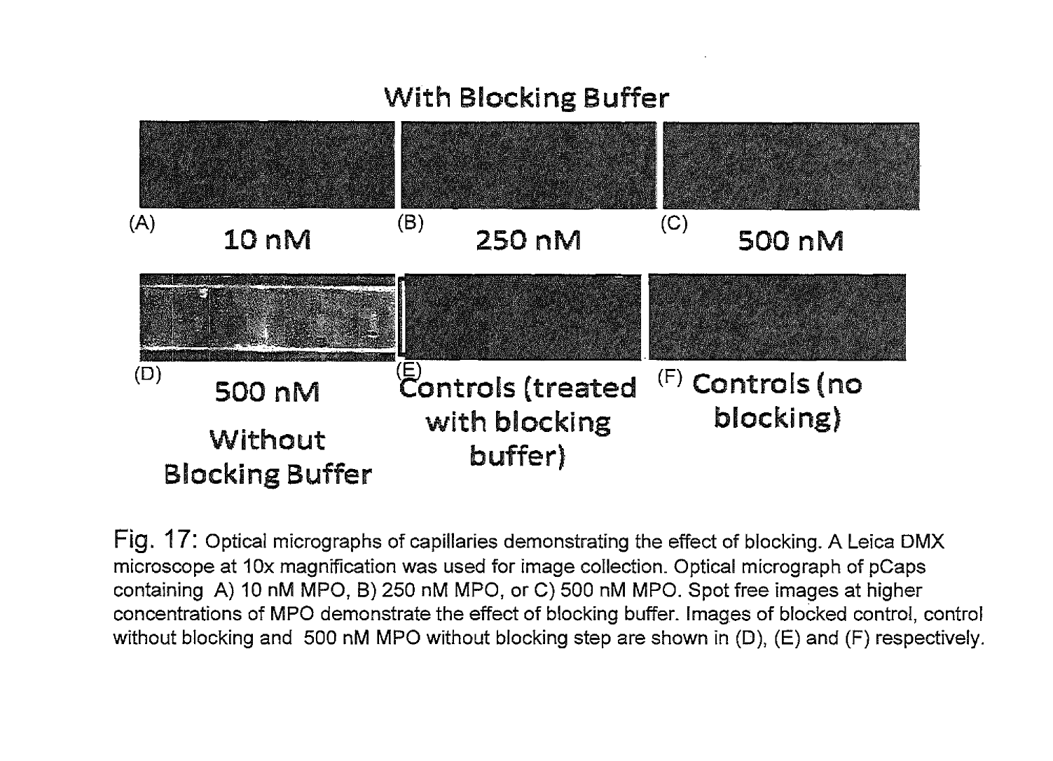

Optical micrographs of capillaries after carrying out the entire QLISA protocol using varying concentrations of MPO is shown in FIGS. 17A through 17C, FIGS. 17A through 17F depict homogenous fluorescence from the capillaries and most importantly an absence of bright spots (indicating aggregation) even at increased concentrations of QDs. The effect of using an optimal concentration of blocking agent is depicted in FIGS. 17A through 17C. FIGS. 17D through 17F represent PMMA capillaries that were not blocked when capturing 500 nM of MPO and control samples that were not blocked during preparation. For reasons of easy visualization, only 500 nM MPO images before and after application of blocking buffer are shown here although similar response was observed with lower concentrations of MPO.

Regions of higher QD intensity (FIG. 17D) in the absence of blocking agent are believe to occur because the functionalization of PMMA capillary leaves vacant sites that can contribute to non-specific binding of Qd-Ab. As most of the biological moieties are charged species at any given pH, it is expected that the target species to bind to the substrate via charge-charge interactions between the target molecule and the sensor substrate. In contrast to these charge-charge interactions, the antigen-to-antibody interaction is very specific, thus biosensor fabrication always includes a step to prevent this nonspecific binding. Functionalization of the capillaries by NaOH results in COOH termination on the PMMA surface which is then used to covalently bind the pAbs to the inner walls of the capillary. Fetal bovine serum (FBS) added to the blocking buffer binds non-specifically to the vacant sites on the inner surface of the PMMA capillary, while leaving the pAb sites with which the MPO can interact. The capillaries can alternatively be functionalized by using plasma or ultraviolet light.

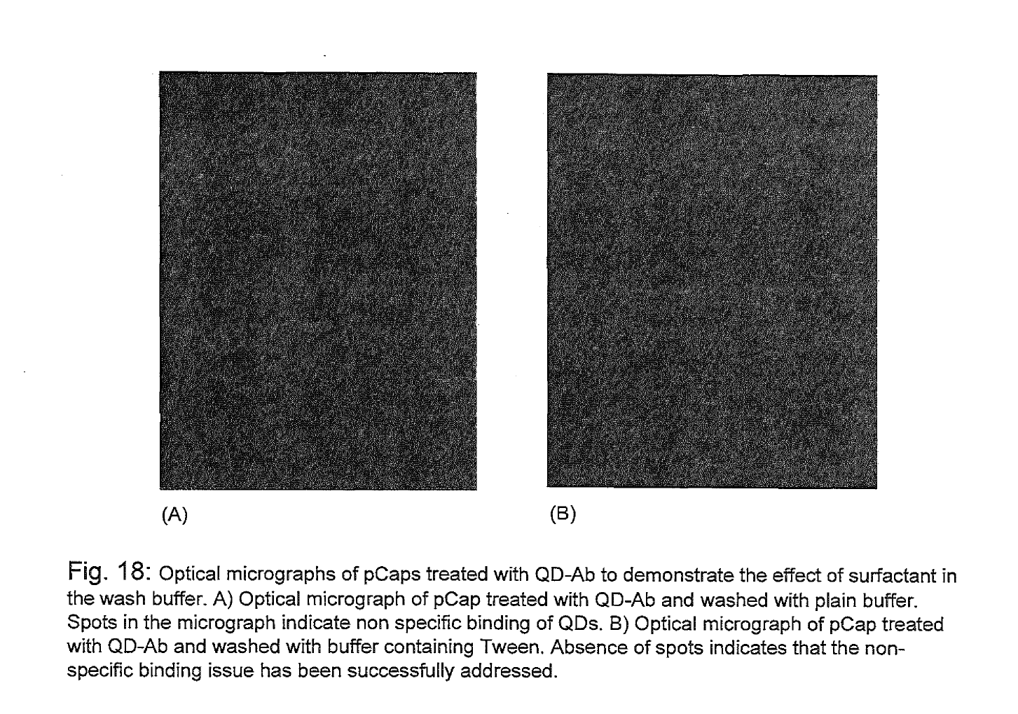

Problems related to non-specific binding are not unique to PMMA capillaries; borosilicate or fused silica capillaries used in immunosensors have exhibited similar issues. Non-specific binding of proteins and enzymes used in the assay can be minimized further by using surfactants during the washing steps. The effect of washing buffer in improving the signal to noise ratio in the QLISA protocol is shown in FIGS. 18A and 18B. Bright fluorescent spots in FIG. 18A correspond to nonspecific binding of QD-Abs to the inner walls of the PMMA capillaries, and they disappeared (FIG. 18B) when a surfactant was included in the wash buffer formulation. Non specific binding of Qd-Ab to PMMA capillary results in bright spots (FIG. 18A), which disappear after washing with wash buffer containing Tween (0.1%) (FIG. 18B).

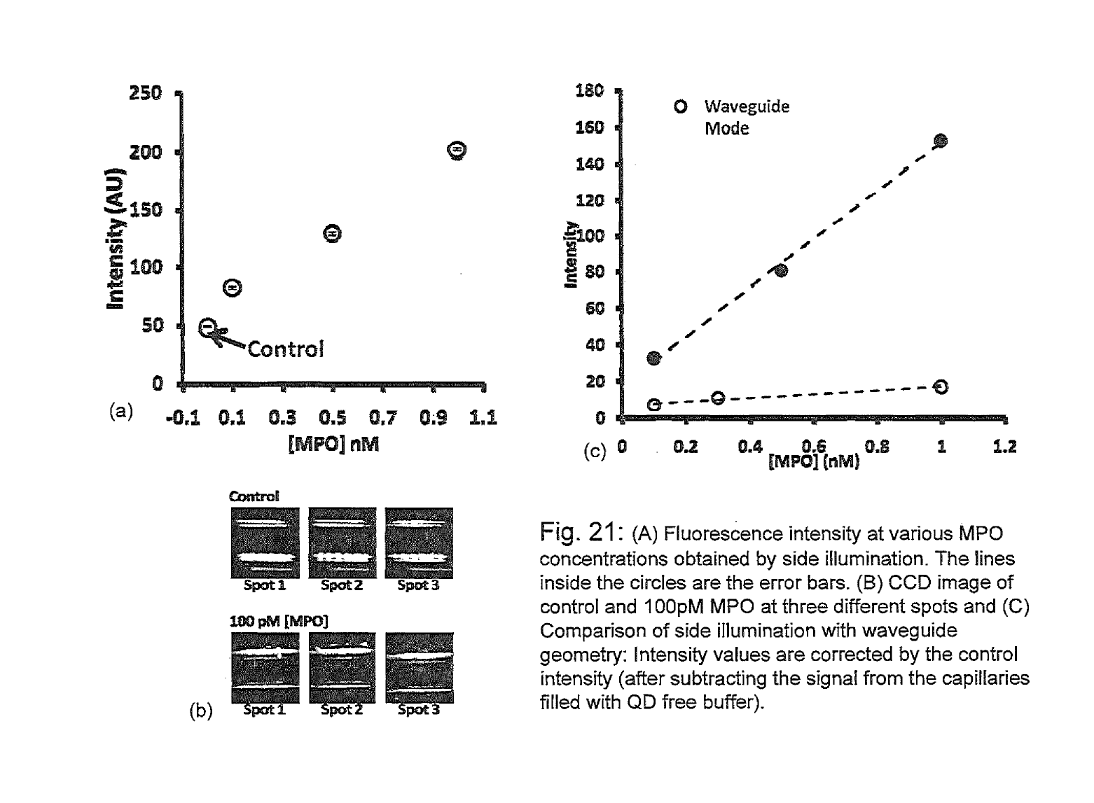

The MPO detection limit was determined as follows. FIG. 21A depicts the performance of the assay in detecting MPO at pico molar concentrations using side illumination. Fluorescence intensity at various locations on a capillary was collected by moving the excitation source that was mounted on a translation stage. This allowed for collection at a rapid rate statistically reliable data from individual capillaries mounted on a custom made spring loaded sample holder, which kept the capillary stretched and aligned.

Representative CCD images (FIG. 21B-100 pM MPO bound capillaries) show improved signal to noise ratio and spot free images that improve the reliability of the data acquisition method. These images were captured at various locations on a capillary and demonstrate uniformity of the fluorescence signal. The bands that appear in the CCD images are result of the architecture of the UV-LED itself. FIG. 21C summarizes the change in fluorescence intensity as a function of MPO concentration in both systems and demonstrates clearly the advantage of the side illumination method. The intensity of control capillaries, i.e. capillaries filled with buffer but containing no QDs, was subtracted from the intensity of PMMA capillaries to yield the data used in FIG. 21C.

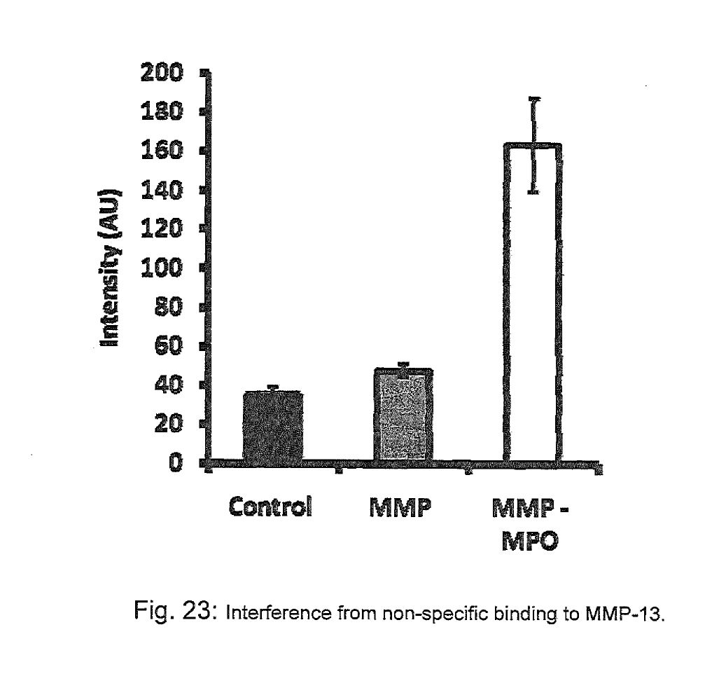

The QLISA protocol was followed to detect MPO in animal stool samples and false positive conditions were simulated by adding Matrix Metalloproteinase (MMP-13) to the tests. MMP-13 is a marker that may be expressed in the case of inflammation and could via non-specific binding lead to false positive results for MPO. Our results indicate possible interference from high levels of MMP-13 (1 nM) still however, well below 10% of the intensity of MPO itself, as shown in FIG. 23. This predicts the specificity of the protocol to be above 90%. This type of work must be done with other key markers in the IBD panel to further establish the specificity of the QLISA protocol.

### Animal Samples

It is known that myeloperoxidase (MPO) is present in stool samples of animals with symptoms mimicking the human Irritable Bowel Disease (IBD). MPO was extracted from stool samples by following the protocol previously established in the art. Briefly, weighed stool samples were digested with an extraction buffer (1×PBS, pH 7.4) supplemented with 12 mM EDTA, 1% Fetal Bovine Serum (FBS), Protease Inhibitor (consisting of AEBSF 0.2 mM, E-64 1.4 μM, Bestatin 13 μM, Leupeptin 0.09 μM, Aprotinin 0.03 μM, EDTA 0.1 mM), 20% Glycerol and 0.05% Tween 20, for 15 minutes at 4° C. The digestion step was followed by homogenization at 5000 RPM till a stable suspension could be obtained, Digestion of the homogenized sample was allowed to continue for 15 minutes at 4° C., followed by centrifugation at 14000 RPM at 4° C. for 30 minutes. The supernatant separated from the centrifuged sample was used in the data disclosed herein. Spiking experiments were carried out by adding 1.0 nM MPO to the stool extract.

The next step in validating the MPO bioassay developed was to test its performance in a more complex system simulating clinical samples. Presence of various biologically relevant moieties that are present in the animal stool samples is a major concern in developing a sandwich assay since the entire assay relies on the specificity and cross reactivity of the polyclonal (capture antibody) and the monoclonal antibody (reporter molecule) towards the antigen. Sensitivity of the QLISA protocol thus depends on the sensitivity and specificity towards one another in the pAb/MPO/QD-Ab sandwich. Although chemistry optimization steps were taken to minimize nonspecific interactions between the sensor substrate and the analyte, it is imperative that the robustness of the protocol and the device be evaluated with actual samples rather than solutions of the antigen.

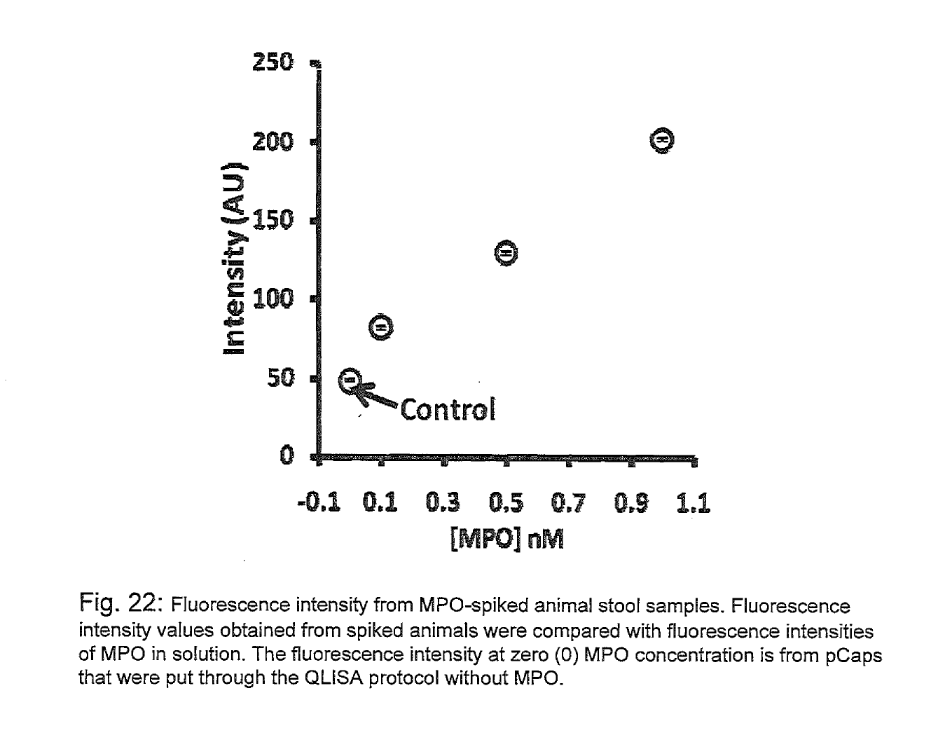

The QLISA protocol disclosed herein was tested in spiked animal stool samples to evaluate its ability to detect MPO at trace levels in biological samples. Stool samples from disease-free mice were collected and prepared as described above. The extract obtained served the purpose of being the control and MPO in solution (1.0 nM) was added to the extract (spiking) from the stool samples. The stool sample extract was spiked such that the final concentration of MPO in the extract was 1, 0.5 and 0.1 nM respectively. In order to get an extract with 1 nM external MPO, 10 μL of external MPO (10 nM) was added to 90 μL of the stool extract and so on. In the mouse model, before inducing disease, the animals bear no MPO in their stools. Intensity values of MPO obtained from the spiked stool extracts were then compared with the intensities from MPO solution. This served as an appropriate test bed for understanding and identifying influence from unknown variables (mostly non-specific binding) that can result in false positive or false negative results. FIG. 22 shows the fluorescence intensity data from stool samples collected from the animals along with data from MPO in solution (standard curve). Spiked stool samples exhibit response that is similar to that of MPO in solution, illustrating the specificity of the antigen/antibody complex and the robustness of the QLISA protocol. The intensity value obtained from the stool sample that does not contain any MPO is essentially the same as the control PMMA capillary of the MPO solution set, indicating the absence of non-specific interaction between the capture antibody and/or the mAb. This was further confirmed by the t-test results, p>0.994, which indicate that the two data sets are identical. The MPO-spiked animal data affirm the expectation that the QLISA bioassay protocol in a full disease model to quantify the presence of MPO in stools and its correlation to clinical disease activity indices.

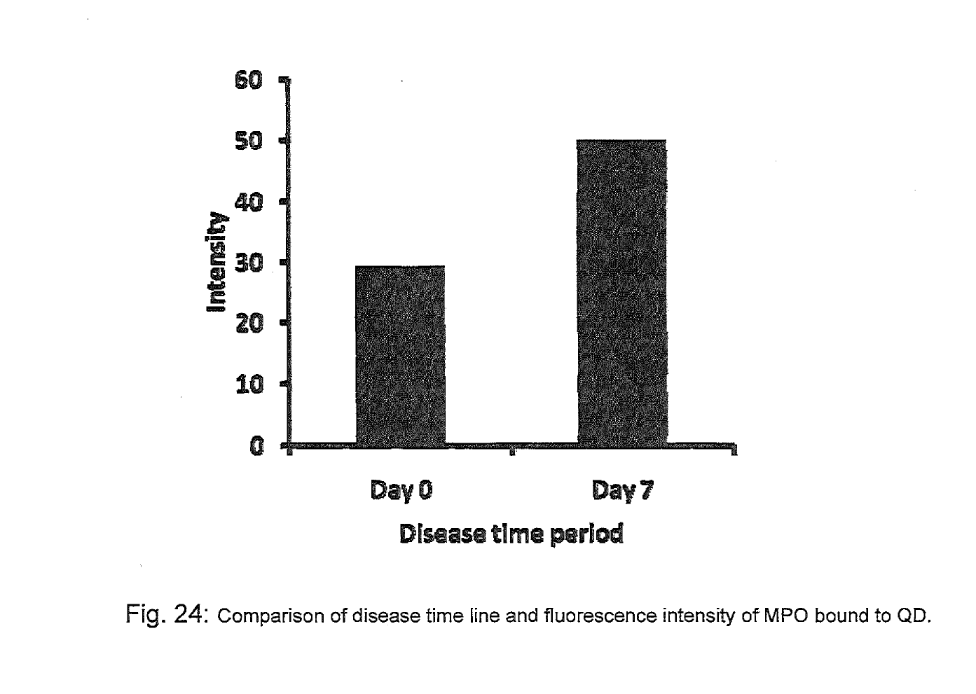

The QLISA protocol was tested on an animal model simulating ulcerative colitis. In particular, the QLISA protocol was tested with animal stool samples to evaluate its ability to detect MPO at trace levels in biological samples. It is known that that MPO level in mice do increase substantially after inducing inflammation by DSS (dextran sodium sulfate) the animals. Our studies therefore compare the level of MPO in stool samples on day 7 and day 0 as shown in FIG. 24. On day 7 the disease is obvious from clinical symptoms, while on day 0 the animals are healthy since DSS feeding has not yet started to induce the disease. The level of MPO (estimated by the QLISA protocol) seems to be almost doubled on day 7 compared to day 0. These results are consistent with previously published work on differences in MPO between diseased and healthy subjects. We have therefore established the capability of the QLISA assay to detect MPO in biological samples and differentiate diseased (day 7) from healthy (day 0) MPO levels.

### Fluid Handling Unit and Multiple Capillary Holder

In one embodiment, streamlined sample preparation and measurement processes allow for simultaneous handling of multiple samples and minimization of errors. Accordingly, a fluid handling unit is provided to capture MPO (10 nM) following the QLISA protocol, in a unit with 12 capillaries in series. Further, an automated mechanical positioning system is provided for the capillaries such that the fluorescent signal from 12 capillaries can be collected by the optical reader in a single pass. Still further, sensitivity of the system is enhanced for detecting MPO in solution and in animal samples and compared with a single capillary system. The multiple sample system utilizes off-the-shelf components for fluid handling, inexpensive high power UV LEDs with extended life (>10,000 hours) for exciting the QDs, and a modular detection system.

Capture antibody immobilized PMMA capillaries attached to a 18 gauge needle are used to capture and detect MPO. This arrangement facilitates fast translation of the technology to a commercialization stage. A computer controlled metering peristaltic pump forces fluids in and out of the capillary tubes. A positioner having two linear stages (XY plane), each with maximum travel distance of about 6 to 8 inches, is used to position the end of the capillaries over reservoirs.

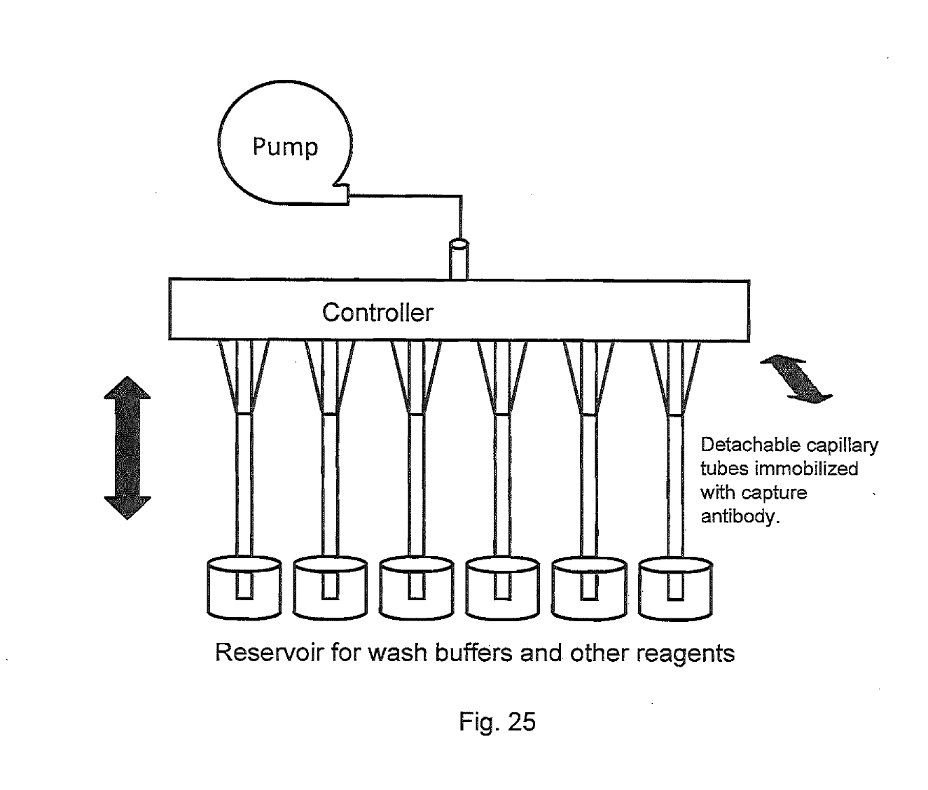

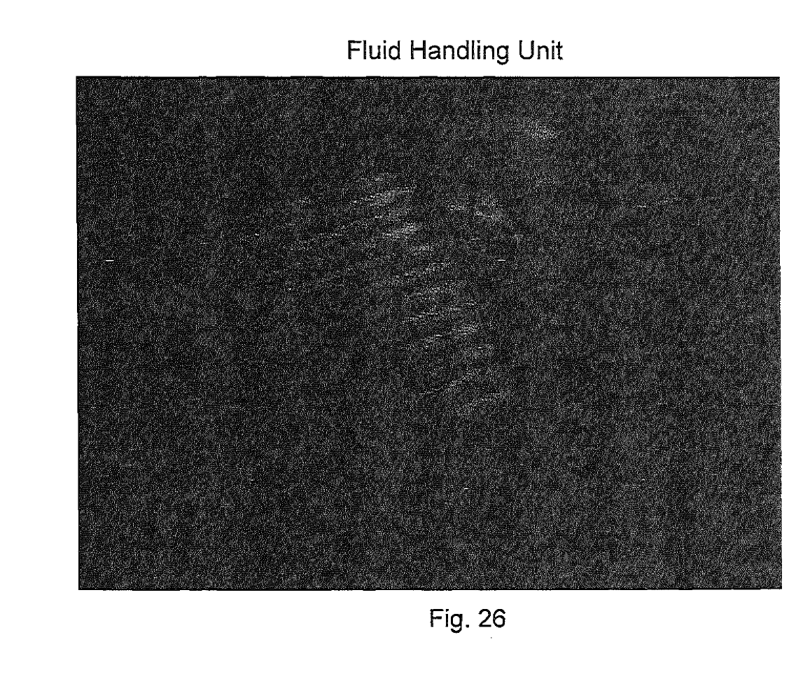

As depicted schematically in FIG. 25, a fluid handling unit holding multiple PMMA capillary tubes can be used to process the QLISA protocol in a plurality of samples simultaneously. An exemplary prototype of the fluid handling unit with 12 detached PMMA capillaries is shown in FIG. 26. The optical system remains the same as that described above with respect to FIGS. 6A and 6B. In this system, UV LEDs (80 mW, λₘₐₓ=405 nm) from Nichia Inc., act as the excitation source, and a cylindrical mirror (f=6.48 mm) is used to collect and focus the fluorescent signal onto a monochrome CCD camera. PMMA capillaries used in the prototype act as fluid conduits and also as waveguides for the excitation of photons, enabling collection of the fluorescence emission signal at a 90 degrees angle to the excitation source.

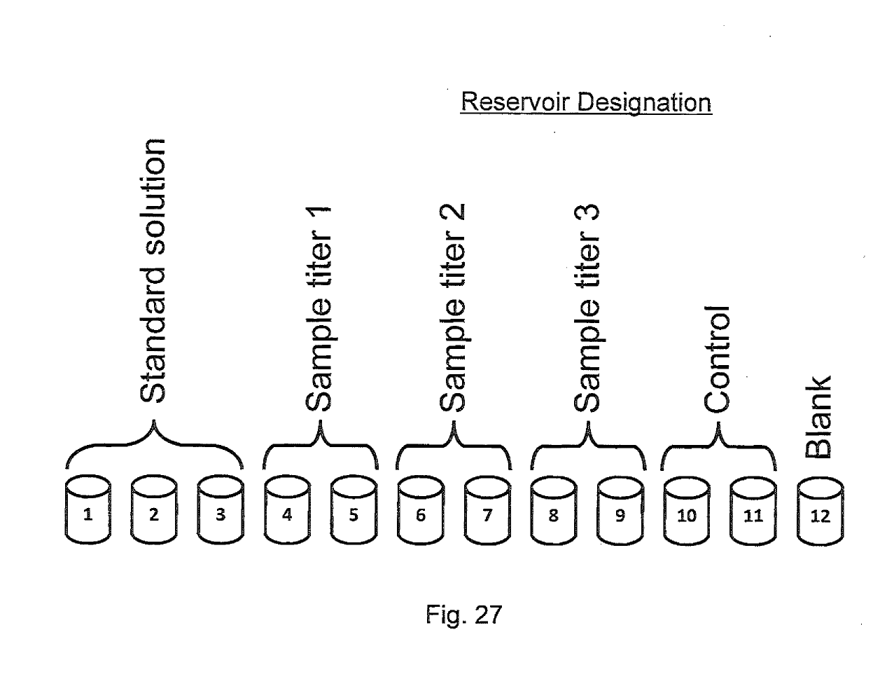

Each capillary tube can be designated for a particular purpose. For example, in the embodiment depicted in FIG. 27, the purpose of all 12 capillaries is shown schematically. More specifically, the fluid handling unit has 12 capillaries in series: three for standard solution (MPO of different known concentrations, e.g., 0.1 nM, 1 nM, and 10 nM), three titer capillaries with duplicates for analyzing the sample, two capillaries for controls (protocol carried out without MPO), and one capillary for spare. Parameters such as flow rate, temperature, and incubation time during MPO capture and detection can be optimized to identify the shortest time required for capturing the antigen. At the end of the capture reaction, the capillaries are detached from the manifold and the fluorescence intensity can be measured using the described optical system.

Transporting the capillaries between reservoirs can be carried out manually, or can be automated. Automated mechanical positioning of the capillaries allows the fluorescent signal from the 12 capillaries to be collected by the optical system in a single pass. An automated version of the QLISA method adapted to process multiple samples simultaneously has been dubbed “Automated Microliter ImmunoSorbent Analysis” (AMISA). A feedback mechanism for positioning the capillaries both in the focal plane of the collection optics and in the focal plane of the excitation source is necessary.

A disposable MPO capture substrate for use with a QLISA process has been developed by immobilizing capture antibodies. Briefly, sodium hydroxide treated PMMA capillaries (250 μm ID, 500 μm OD) affixed to glass capillaries of 600 μm ID serve as the MPO capture substrate. The glass capillaries serve as a mechanical support for the PMMA capillaries. Glass/PMMA composite structures are then attached to the hub of the 18 gauge needle after trimming the cannular shaft. The flange at the end of the hub facilitates the attachment of the structure to the barrel at the ports of the fluid handling unit.

Capillary force is typically sufficient for the uptake of solutions into the capillary. However a positive pressure can be also set up at the hub to force the solutions out of the capillary. Negative pressure at the hub ensures that liquid is drawn to a predetermined level inside the capillary, if capillary force alone is found to be inadequate. A four way solenoid valve and a peristaltic pump are used to cycle the pressure at the hub and provide fresh reagents to the MPO capture sites. A 96 well plate serves as the reservoir for wash buffer, blocking agents, capture and detecting antibody, and the sample. The fluid handling unit can be automatically cycled through samples using combination of X and Z axis linear stages.

The excitation source comprises a high power UV LED from Nichia Inc, a forced cooling system, and focusing optics. Commercially available electronic drivers and cooling system are used in the LED source system, and short focal length fused silica or quartz planoconvex lenses are used to focus the UV light from the LED. The fluorescence emission from the QD solution taken in the glass/PMMA composite structure is used to determine the distance between the tip of the capillary and the light source as well as the placement of the capillary tube at the optical axis of the cylindrical mirror. A CCD camera focused on the capillary tube functions as the signal collection device. Custom Matlab routines for image analysis are used to analyze and compute the intensity of the fluorescent signal. A linear stage with feedback control mechanism completes the task of acquiring data from a set 12 capillaries, attached to the fluid handling unit. Commercially available motion control packages from Labview are used to control the linear stage and placement of individual capillaries at the focal plane of the mirror.

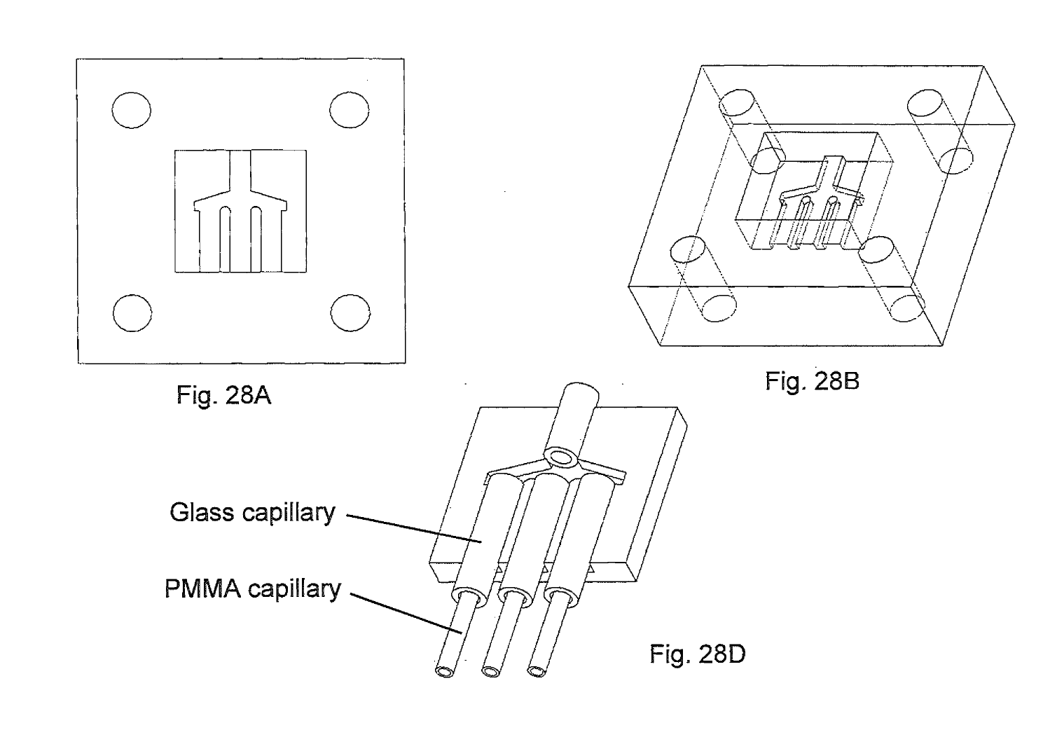

A castable multiple capillary holder has been designed for handling a plurality of capillaries simultaneously. As depicted in FIGS. 28A through 28D, a mold is provided for making an embodiment of the multiple capillary holder adapted to handle three capillaries simultaneously. By using a multiple capillary holder, processing can be reduced and triplicate experiments can be run in parallel.

As depicted, the multiple capillary holder includes one inlet port for charging the capillaries and multiple outlet ports to which capillaries can be coupled. The inlet port can be coupled to a pump via standard fluid couplings or glued to a 21 gage or 26 gage needle. A holder with a needle as the inlet port has an advantage of being able to use hassle-free luer lock type connectors. FIGS. 28A through 28C depict a silicone master mold that has been fabricated to form the holder.

Each of the inlet port and outlet ports is preferably made integral with a glass capillary. The master mold has raised portions, as indicated in FIGS. 28A and 28B, such that a holder can be formed in relief, as shown in FIG. 28C. The multiple capillaries are then placed into recesses in the holder and permanently coupled to each other and the holder by applying an epoxy, as shown in FIG. 28D.

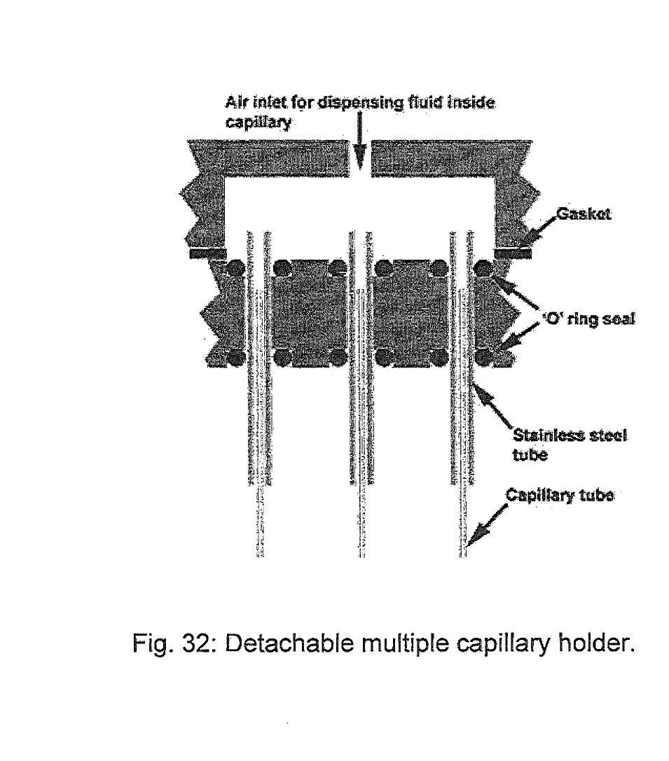

FIG. 31 depicts a further exemplary embodiment of a sampling manifold for holding a plurality of capillaries each supported by stainless steel sleeves. FIG. 32 depicts a detachable multiple capillary holder that allows the capillaries to be loaded and dispensed simultaneously via a common air header at one end of the capillaries.

Improving Stability of Quantum Dots by Altering their Microenvironment

In many instances, particularly when testing of a sample cannot be completed immediately, it is important to stabilize the fluorescence of quantum dots, i.e., to reduce the reduction of fluorescence intensity of quantum dots, during storage and imaging. Based on the work described herein, the degradation of fluorescence of QDs over time appears to be the result of a combination of mechanisms. As disclosed, various fluorescence stabilizing solutions can be formulated having one or more characteristics that serve to minimize decay of QD fluorescence intensity, or in some case to increase fluorescence intensity, over time.

For example, continued exposure of quantum dots to solutions of medium to high ionic strength results in a continuous loss of their fluorescence over a period of time. Additionally, the loss of fluorescence depends not only on the ionic strength but also on the pH of the solution. Some commercially available quantum dots exhibit higher stability in solutions above pH 8.0 and loose their intensity rapidly in mildly acidic conditions (below pH 7.0). For those QDs, the rate of loss of intensity increases with decrease in pH. Other commercially available QDs appear to have a maximum loss of fluorescence around approximately 7.5 pH. Also, while the degradation of fluorescence was detected with all QDs tested without exposure to a fluorescence stabilizing medium, the rate and amount of loss of fluorescence can vary to some extent depending on which commercially available QD is tested, even when testing QDs that are believed to be structurally the same.

As described below, methods disclosed herein rely on altering the local environment of the quantum dots by replacing the buffered solution or medium in which the QDs are typically stored with appropriate liquids of low or zero ionic strength, or by conditioning the liquid in which QDs are stored by the addition of an antioxidant such as vitamin E. Other known substances with antioxidant properties can be used to stabilize the fluorescence of QDs in a solution or medium, including but not limited to phenolic antioxidants (sterically hindered or not), NOR chemicals, lactone, hydroxylamine, antioxidant enzymes such as superoxide dismutase or species that can quench free radical damage, and other antioxidants known in the art such as those sold commercially by Ciba. As with the vitamin E described herein, the antioxidant used should be water soluble so as to be able to disperse in the QD-containing medium. Different antioxidants reduce the loss of fluorescence and may provide additional means of stabilization.

All work herein was performed with CdSe/ZnS quantum dots which have a CdSe core with a ZnS protective layer. Quantum dots tested include those manufactured by Ocean Nanotech LLC (“Ocean Nanotech”) and by Invitrogen Corporation (“Invitrogen”). Both manufacturers use polyethylene glycol as a protective group. However, based on observed differences in optical and other properties of QDs supplied by the two manufacturers, as well as data related to the decay of fluorescence intensity, there appears to be a proprietary difference between the two groups of QDs. Ongoing stability studies continue using free radical scavengers, and are investigating a non-polar polymeric system with varying refractive index values and their effect in reducing the loss of fluorescence from quantum dots.

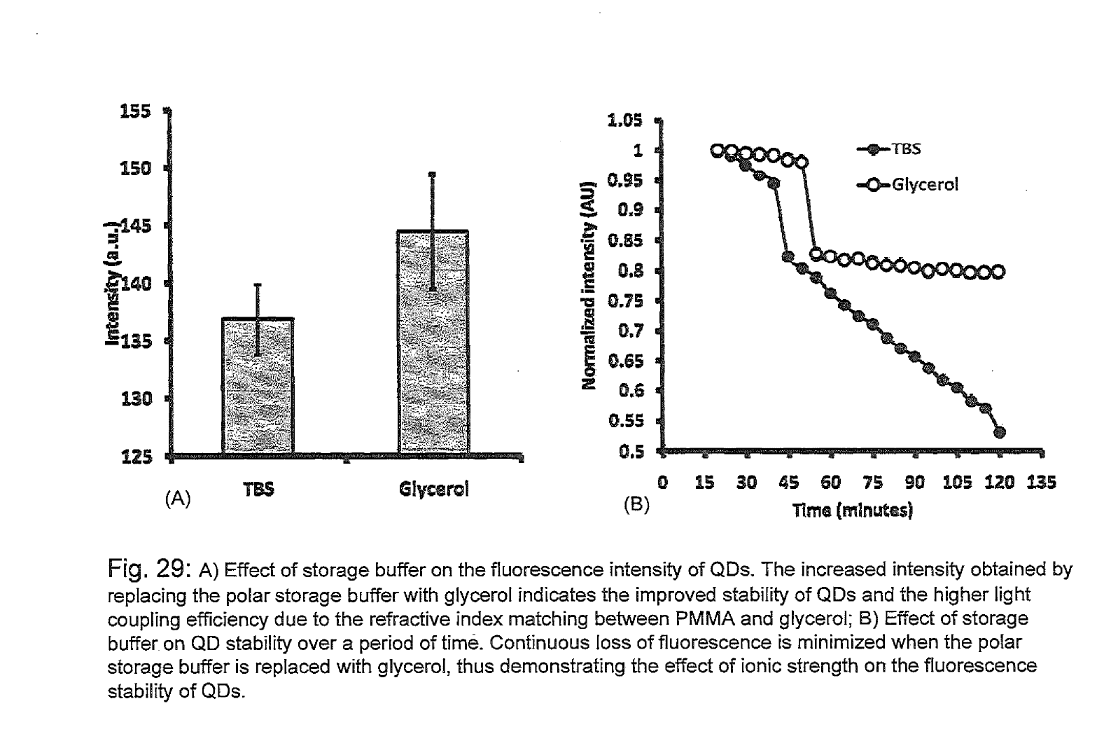

FIGS. 29A and 2913 compare the effect of ionic strength on the intensity and stability of the QD fluorescence signal over periods of time. MPO in solution (0.5 nM) was used in this experiment and the effect of ionic strength on the intensity of the QDs was investigated by replacing the wash buffer with glycerol. It is understood that other buffers of that are similarly non-polar or weakly polar could be used. Increase in fluorescence intensity was observed when the storage buffer (Trs-Buffered Saline, TBS) was replaced with glycerol (FIG. 29A).

The photo stability of QDs and hence their fluorescence yield depends heavily on their local environment, especially the ionic strength of the local environment. This effect has been utilized to fabricate optical metal ion sensors and intracellular pH sensors. Therefore, replacing the buffer with a non-ionic solution such as glycerol increases the signal to noise ratio, as observed in FIG. 29A, by two possible means. First, with glycerol the system is deprived of ions that would otherwise destabilize QDs, and therefore the maximum attainable quantum yield of QDs can be attained. Second, scattering is minimized by confining more of the photons in the walls of the capillary because the refractive index of glycerol (1.398) is closer to that of PMMA (1.491) than the storage buffer (˜1.33). As expected, PMMA capillaries with glycerol display a marked increase in intensity, which is thought to be due to a combined effect of increased photon coupling and its role in stabilizing the fluorescence itself of the quantum dots. In order to substantiate that ionic strength of the local environment of QDs is a causal reason for the difference in fluorescence intensity between the two liquids, fluorescence intensities from images of PMMA capillaries with storage buffer and with glycerol were collected over a period of 2 hours and compared.

FIG. 29B compares the effect of ionic strength on the stability of QD fluorescence over a period of 2 hours. A steady decline in signal intensity was observed when QDs were exposed to an environment of high ionic strength (Trs-Buffered Saline, TBS), leading to a loss in intensity of nearly 50%. However, replacing the wash buffer inside the capillary with glycerol, resulted in minimal loss of signal intensity (about 15% loss versus about 50% loss). In both environments there is a fast decay in signal intensity within the first 60 minutes. This experiment was conducted by collecting images every five minutes and the QDs were excited only during a brief period of image acquisition mode. This ensures that the QDs or the PMMA capillaries were not heated by the excitation source during the course of the experiment. Replacing the storage buffer provides a critical advantage for the reliability of the assay, as shown in FIG. 29B.

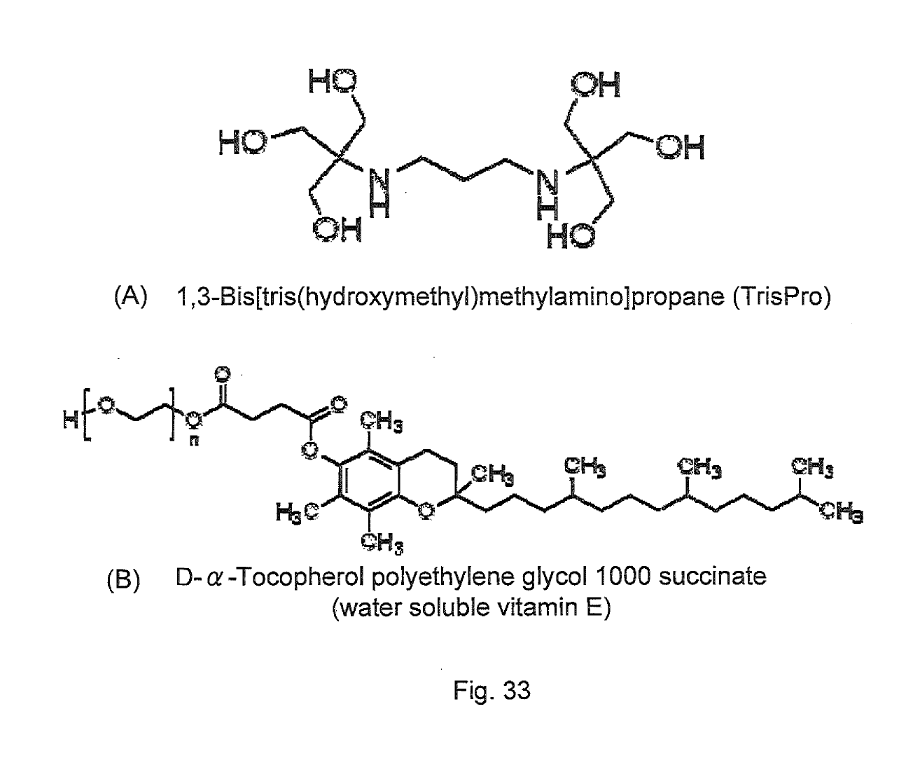

In another set of experiments, water-soluble vitamin E (D-a-Tocopherol polyethylenhe glycol 1000 succinate, Chemical Abstracts Services Reference Number 9002-94-4, shown chemically in FIG. 33B) was dissolved in a water-based buffering agent (1,3-Bis[tris(hydroxymethyl)methylamino]propane (“TrisPro”), shown chemically in FIG. 33A), and the effect on fluorescence decay over time was measured. The water solubility of the vitamin E is about 1 gram per 10 milliliters, or about 10%. Solutions having a pH in the range of about 6 to about 9.5 were tested, and data is presented herein for solutions of pH at about 6.5, about 7.5, and about 8.5.

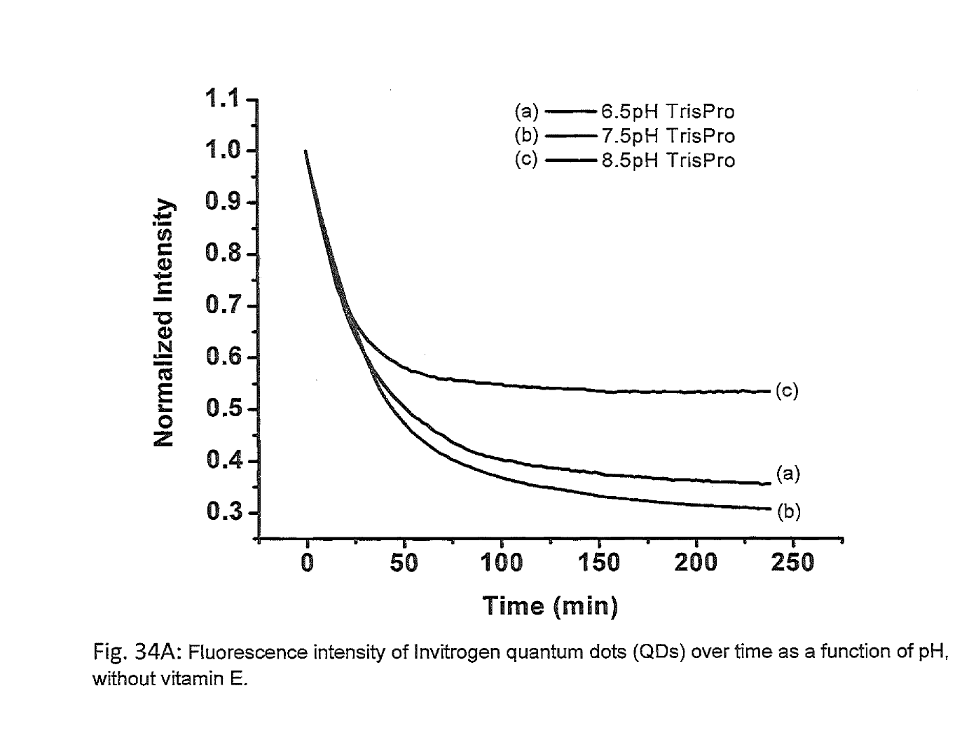

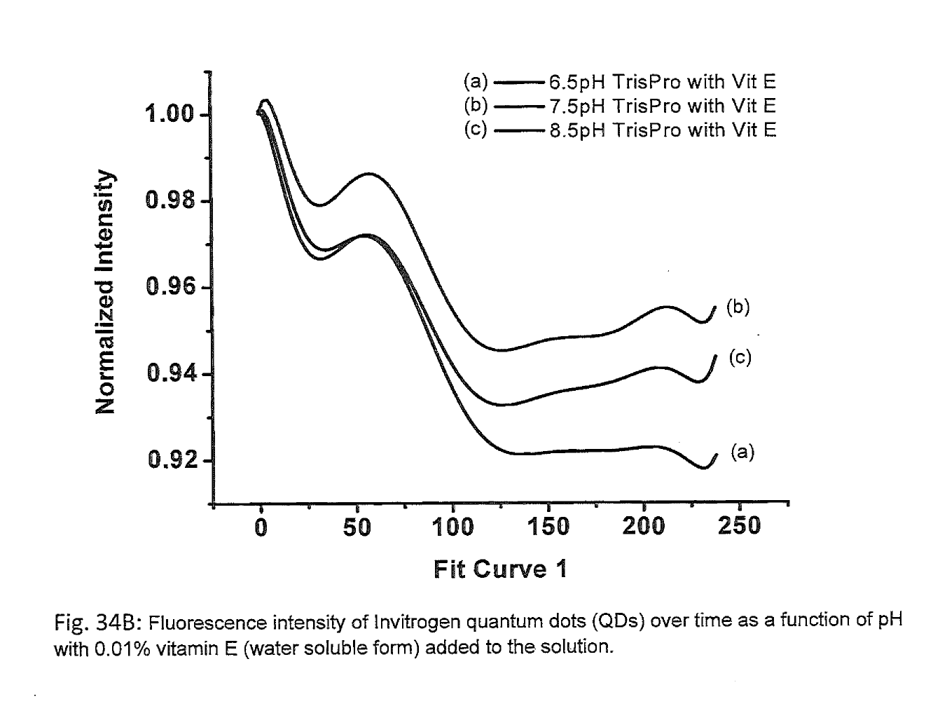

FIGS. 34A and 34B compare the decay of fluorescence intensity over time using Invitrogen QDs in a solution without vitamin E versus a solution including about 0.01% vitamin E. Note that the Y-axis scale differs on the two graphs, with FIG. 34A ranging from about 0.25 to 1.0 and FIG. 34B having a much tighter span of about 0.91 to 1.00. It can be seen that for all three pH levels tested, the decay of fluorescence intensity was dramatically decreased in the vitamin E medium as compared with the non-vitamin E medium. In particular, at a pH of 6.5, the fluorescence of QDs in the vitamin E solution decreased by only about 8% after 240 minutes as compared with a decrease of about 60% in the non-vitamin E solution. Similar, at a pH of 7.5, a decrease of only about 5% in fluorescence intensity was observed at 240 minutes in the vitamin E solution as compared with a decrease of over 60% in the non-vitamin E solution. At a pH of 8.5, an approximately 40% decrease in fluorescence intensity after 240 minutes in the non-vitamin E solution was reduced to a decrease of only about 7% in the vitamin E solution.

Additionally, the decay of fluorescence intensity was rapid in the non-vitamin E solutions, with the QDs losing between about 40% and about 55% of their intensity in the first 50 minutes, whereas the fluorescence intensity in the vitamin E solutions was maintained at above about 97% of the original intensity after 50 minutes, regardless of the pH of the solution. Further, by about 125 minutes in the vitamin E solutions, the decay of fluorescence appeared to stabilize and the fluorescence intensity even increased slightly, while in the non-vitamin E solutions, particularly for the solutions at 6.5 and 7.5 pH, the fluorescence intensity continued to degrade for as long as measurements were taken. The amount of response to the presence of vitamin of the same concentration of vitamin E differed depending on the pH of the buffer solution. In particular, in the non-vitamin E solutions, the QDs held at a pH of 7.5 showed the most degradation in fluorescence and the QDs held at a pH of 8.5 showed the least, while in the 0.01% vitamin E solutions, the QDs held at a pH of 7.5 lost the least amount of fluorescence intensity and the QDs held at a pH of 6.5 lost the most.

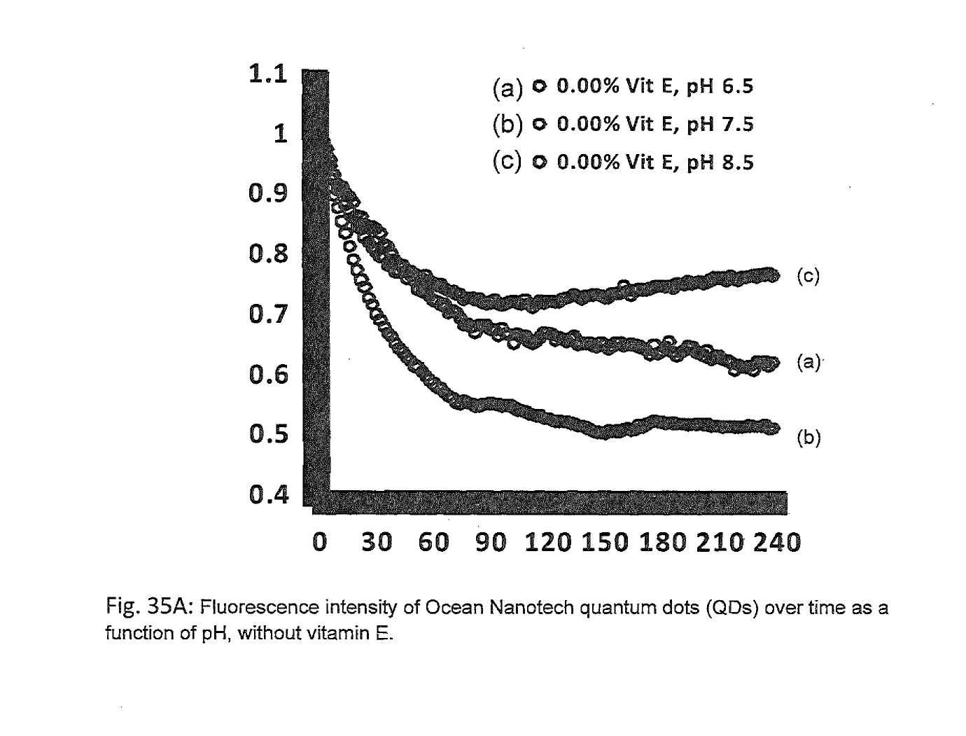

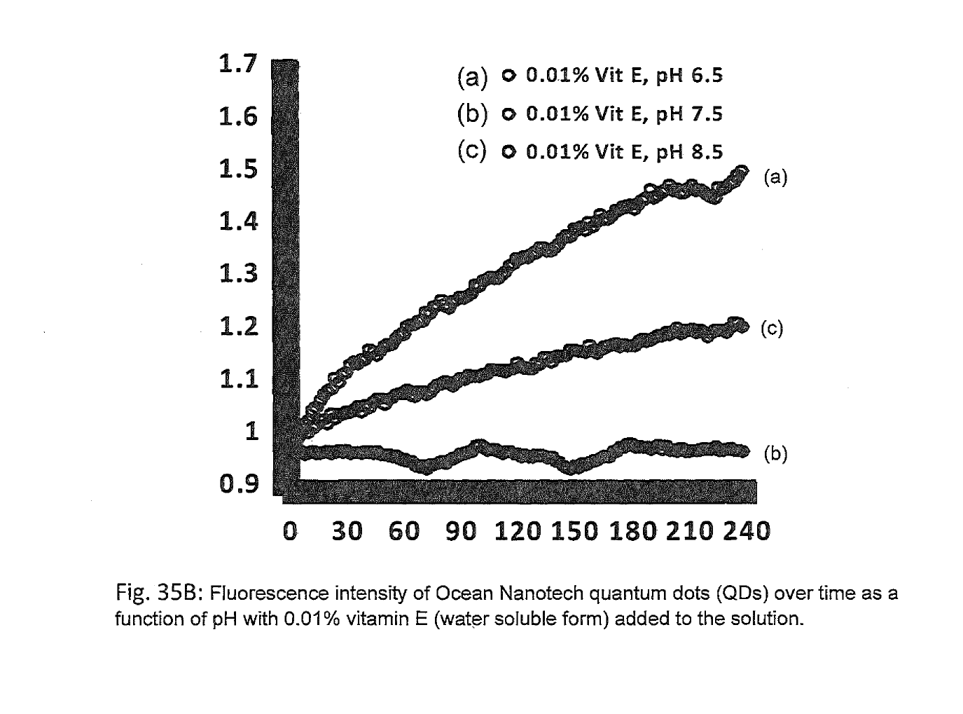

FIGS. 35A and 35B compare the change in fluorescence intensity over time using Ocean Nanotech QDs in a medium without vitamin E versus a medium including about 0.01% vitamin E. At a pH of 6.5, the fluorescence intensity in the non-vitamin E solution decreased by about 35% after 240 minutes while the intensity in the vitamin E solution actually increased by about 50% over the same time period. At a pH of 7.5, the fluorescence intensity in the non-vitamin E solution decreased by nearly 50% after 240 minutes while the intensity in the vitamin E solution remained nearly constant over the same time period. At a pH of 8.5, the fluorescence intensity in the non-vitamin E solution decreased by over 20% after 240 minutes while the intensity in the vitamin E solution increased by about 20% over the same time period. As with the Invitrogen QDs, the amount of response to the presence of the same concentration of vitamin E differed depending on the pH of the buffered solution. In particular, in the non-vitamin E solutions, the QDs held at a pH of 7.5 showed the most degradation in fluorescence and the QDs held at a pH of 8.5 showed the least, which was a similar result to the Invitrogen QDs. However, in the 0.01% vitamin E solutions, the QDs held at a pH of 6.5 had the greatest increase of fluorescence intensity and the QDs held at a pH of 7.5 had the smallest increase (of approximately zero), which was a different result to the Invitrogen QDs. The cause of the different responses to vitamin E at varying pH levels between the Invitrogen QDs and the Ocean Nanotech QDs is the subject of ongoing investigation.

The change of fluorescence of the each of the two types of QDs in a vitamin E solution was relatively consistent regardless the concentration of vitamin E, provided the concentration was equal to or greater than a threshold concentration of about 0.001% vitamin E. It should be noted that lesser concentrations may be effective but were not tested, so that an absolute minimum threshold vitamin E concentration was not definitively determined. The fluorescence intensity of QDs over time was measured in solutions at three different pH levels and at four non-zero vitamin E concentrations, about 0.05%, about 0.01%, about 0.005%, and about 0.001%, as well as a control of 0% vitamin E.

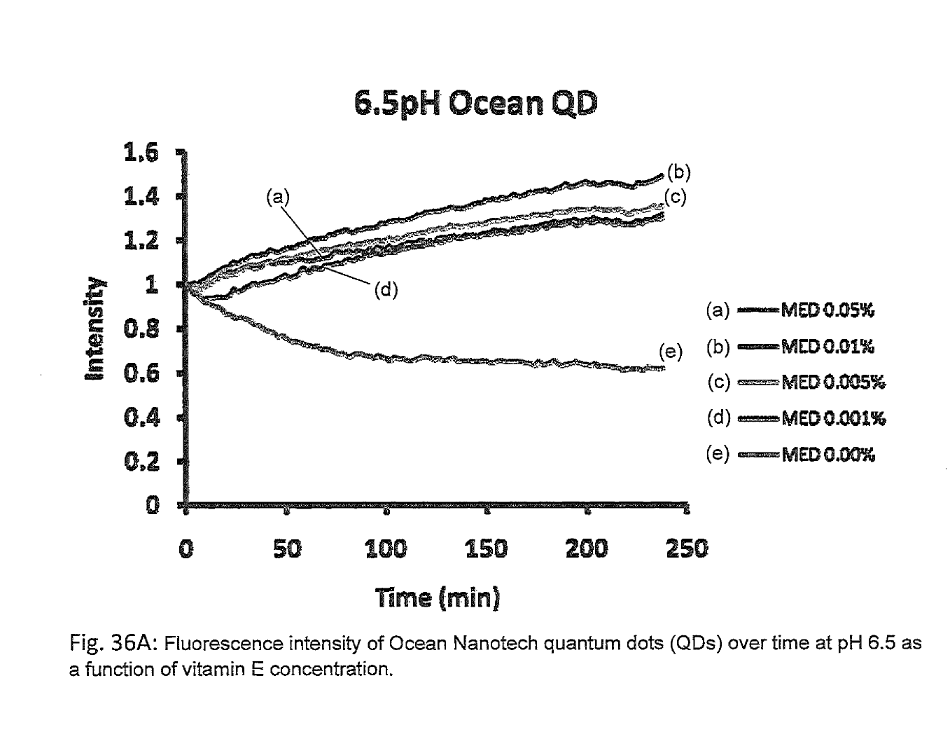

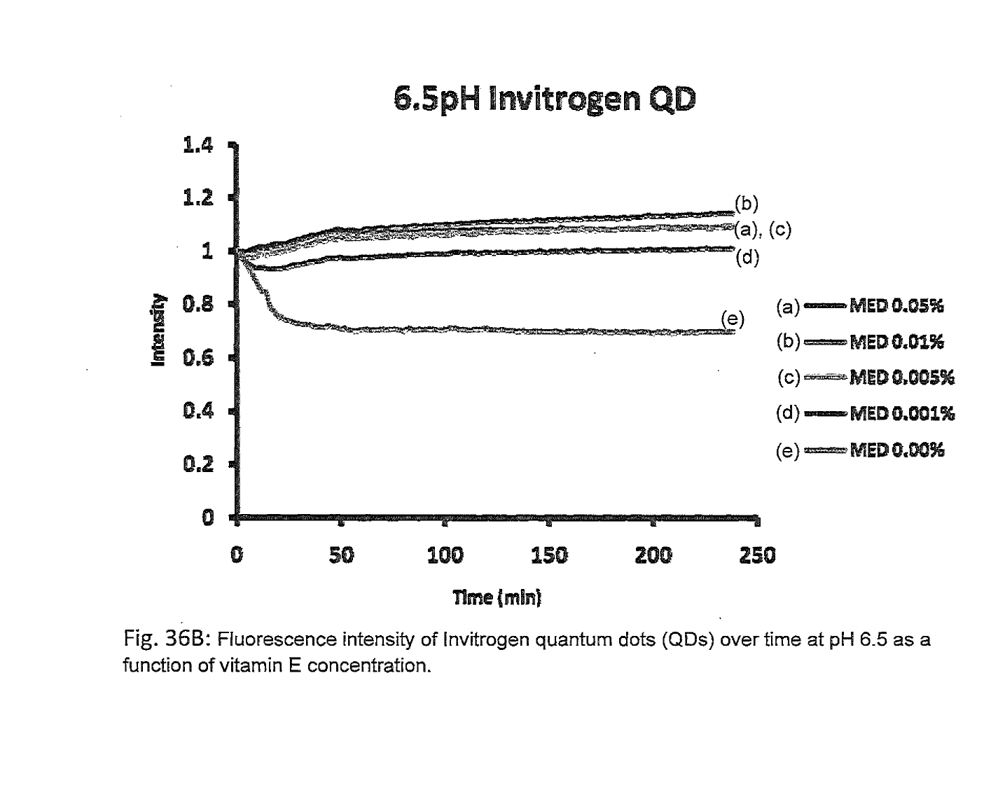

As shown in FIG. 36A, for Ocean Nanotech QDs in a medium of 6.5 pH, the fluorescence intensity increased over 240 minutes by between about 30% and about 50% for the vitamin E solutions, while the intensity decreased by nearly 40% in the non-vitamin E solution. A vitamin E concentration of about 0.01% yielded the greatest increase in intensity, while concentrations of about 0.05%, about 0.005%, and about 0.001% were all similarly effective at improving fluorescence intensity. As shown in FIG. 36B, for Invitrogen QDs in a medium of 6.5 pH, the fluorescence intensity remained about the same over 240 minutes for a vitamin E solution of about 0.001% and increased by as much as about 15% for the other vitamin E solutions, while decreasing by almost 30% for the non-vitamin E solution.

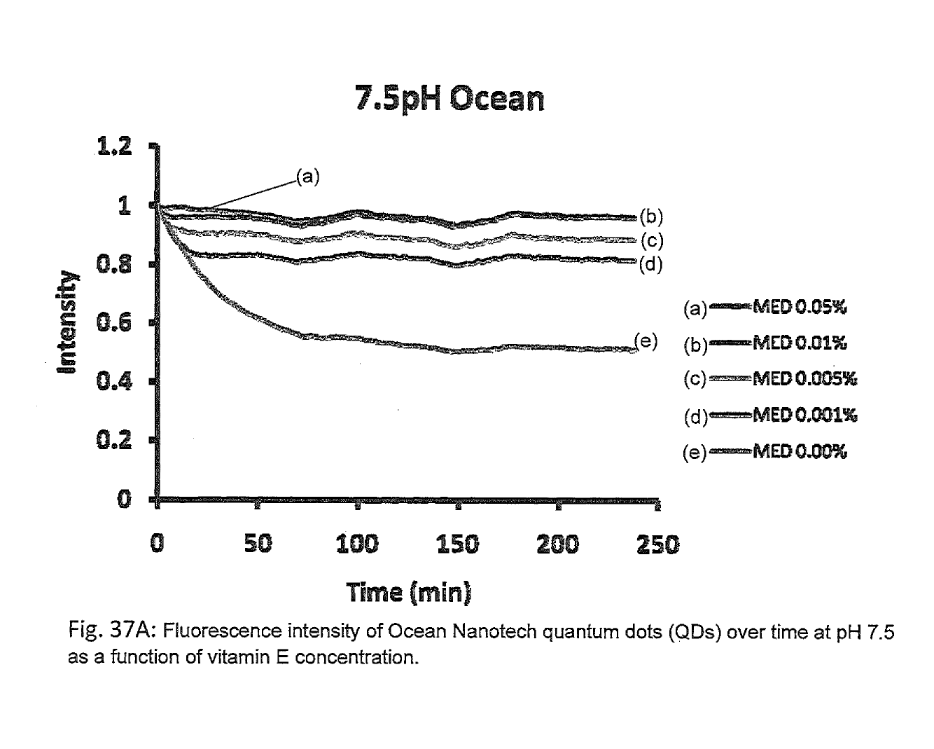

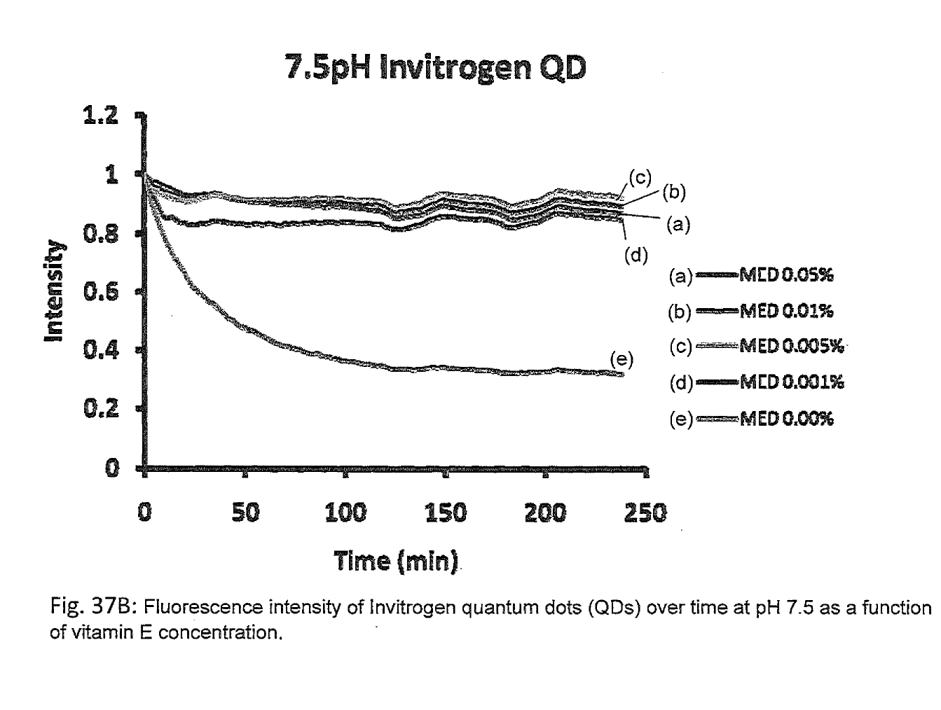

As shown in FIG. 37A, for Ocean Nanotech QDs in a buffer of 7.5 pH, the fluorescence intensity over 240 minutes dropped by less than about 20% in the 0.001% vitamin E solution, by less than about 10% in the 0.005% vitamin E solution, and remained about the same in the higher concentration vitamin E solutions, while the intensity decreased by nearly 50% in the non-vitamin E solution. As shown in FIG. 37B, for Invitrogen QDs in a buffer of 7.5 pH, the fluorescence intensity was reduced over 240 minutes in the vitamin E solutions by between about 5% and about 15%, while decreasing by nearly 70% in the non-vitamin E solution. Thus, across the board, the QDs of both manufacturers fared significantly worse in the solutions of 7.5 pH as compared with solutions of 6.5 pH, although at both pH levels the presence of even 0.001% vitamin E dramatically reduced the degradation in fluorescence intensity, and in some cases increased fluorescence intensity in the 6.5 pH solutions.

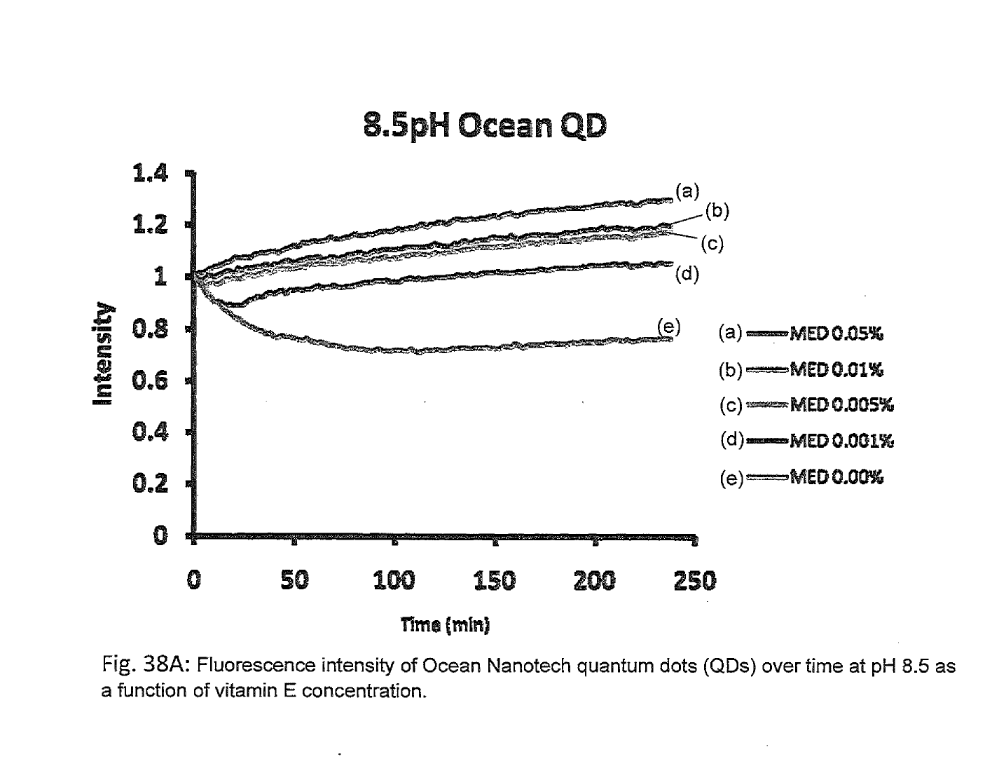

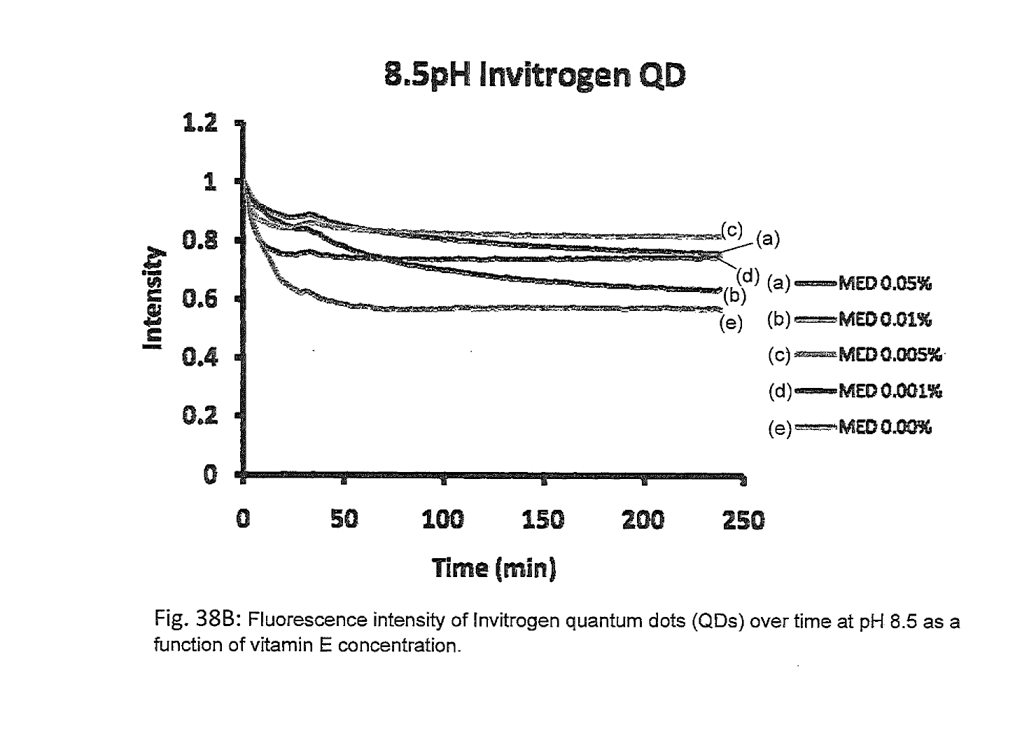

As shown in FIG. 38A, for Ocean Nanotech QDs in a buffer of 8.5 pH, the fluorescence intensity over 240 minutes varied from about the same to an increase of about 30% in the vitamin E solutions. In particular, the intensity dropped initially but recovered to its original level in the 0.001% vitamin E solution, increased by about 30% in the 0.05% vitamin E solution, and increased by about 15% in the 0.01% and 0.005% vitamin E solutions. In contrast, the fluorescence intensity dropped by over 20% in the non-vitamin E solution, As shown in FIG. 38B, for Invitrogen QDs in a buffer of 8.5 pH, the fluorescence intensity was reduced over 240 minutes for every solution, but was reduced less in the vitamin E solutions than in the non-vitamin E solution. In particular, the intensity dropped by about 15% in the 0.005% vitamin E solution, by about 20% in the 0.001% and 0.05% vitamin E solutions, by over 30% in the 0.01% vitamin E solution, and by nearly 40% in the non-vitamin E solution, Accordingly, vitamin E ameliorated the deterioration of fluorescence intensity in a solution of 8.5 pH for the Invitrogen QDs and still was able to cause in increase in fluorescence intensity for the Ocean Nanotech QDs,

### Expansion of QLISA Protocol to Additional Biomarkers

Current available tests (not using QDs or a QLISA protocol) to distinguish IBD from IBS include calprotectin and lactoferrin ELISAs. Calprotectin has been shown to have 89% sensitivity and 96% specificity in differentiating IBD from IBS. Lactoferrin has been identified as a reliable marker for differentiating IBD from IBS (90% specificity and 87-92% sensitivity). More specific tests for distinguishing between Ulcerative Colitis (UC) and Crohn's Disease (CD), are based on the presence of antibodies like perinuclear antineutrophilic cytoplasmic antibody (p-ANCA) and anti-Saccharomyces cerevisiae antibody (ASCA) in serum, and have specificity less than 70%. Presence of lactoferrin and myeloperoxidase (MPO) and their ratio has also been shown to differ sufficiently to differentiate IBD from infectious diarrhea. Thus early detection of MPO could facilitate in differentiating IBD from IBD and also TBD from infectious diarrhea. The currently best available method for Myeloperoxidase detection in stool samples is radioactive labeling. This method is time consuming, requires expensive labeling facilities and could expose patients to radiation risks. Radioactive labeling was used in a 2006 study of MPO to detect inflammation in IBS and collagenous colitis as the only reliable quantitative assay. Therefore, there is a need for a user friendly, fast and inexpensive detection assay. Current immunoassays require 24 hours of laboratory preparation time, whereas a QLISA test can require less than 3 hours.

Ongoing testing is being done to demonstrate the QLISA method in human samples of MPO, lactoferrin and calprotectin, and also to develop a test kit that can measure simultaneously in triplicates these markers including relevant standards and in a semi automated manner. The test kit will include functionalized capillaries with a specific antibody or antigen, special reagents for performing the test (e.g. functionalized QDs, wash buffers), and an optical reader.

Both lactoferrin and calprotectin are antigens similar to MPO and the QLISA protocol disclosed herein and a similar a protocol is expected to be readily applied for detecting lactoferrin and calprotectin in stool samples with appropriate optimization steps to fit the capture and reporter antibodies. Sensitivity and specificity of the QLISA protocol in determining lactoferrin and calprotectin will be evaluated in both solution and human samples. It is clear that both lactoferrin and calprotectin have important predictive value and it is expected that combining them with MPO detection will allow for more accurate initial diagnosis and follow up. The need for measurements of all three highlights the importance of QLISA as a microcapillary assay, capable of measuring all three with minimal sample obtained during routine exams.

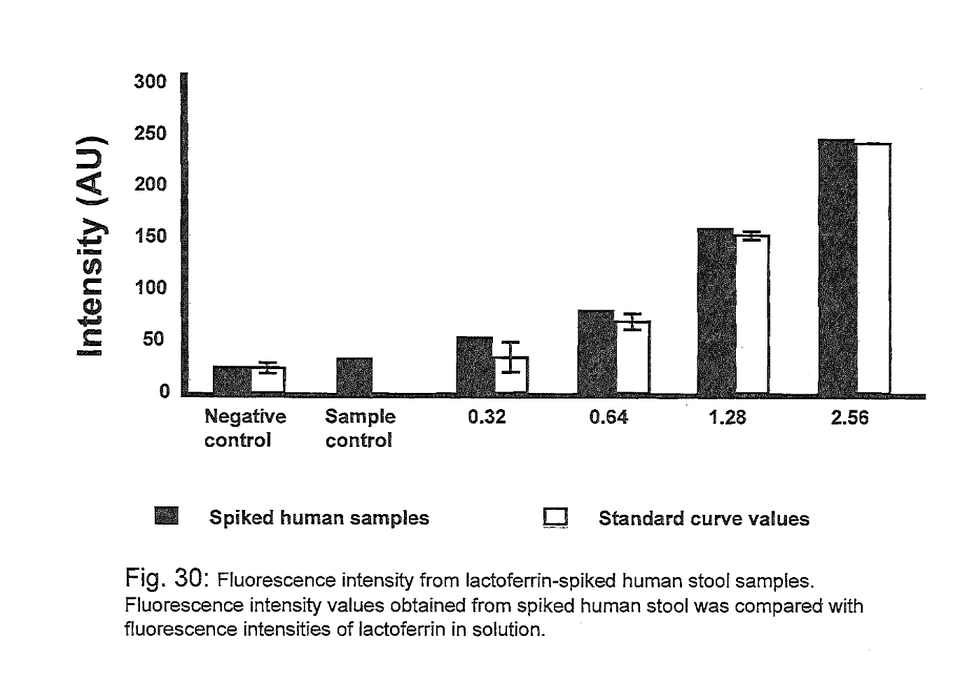

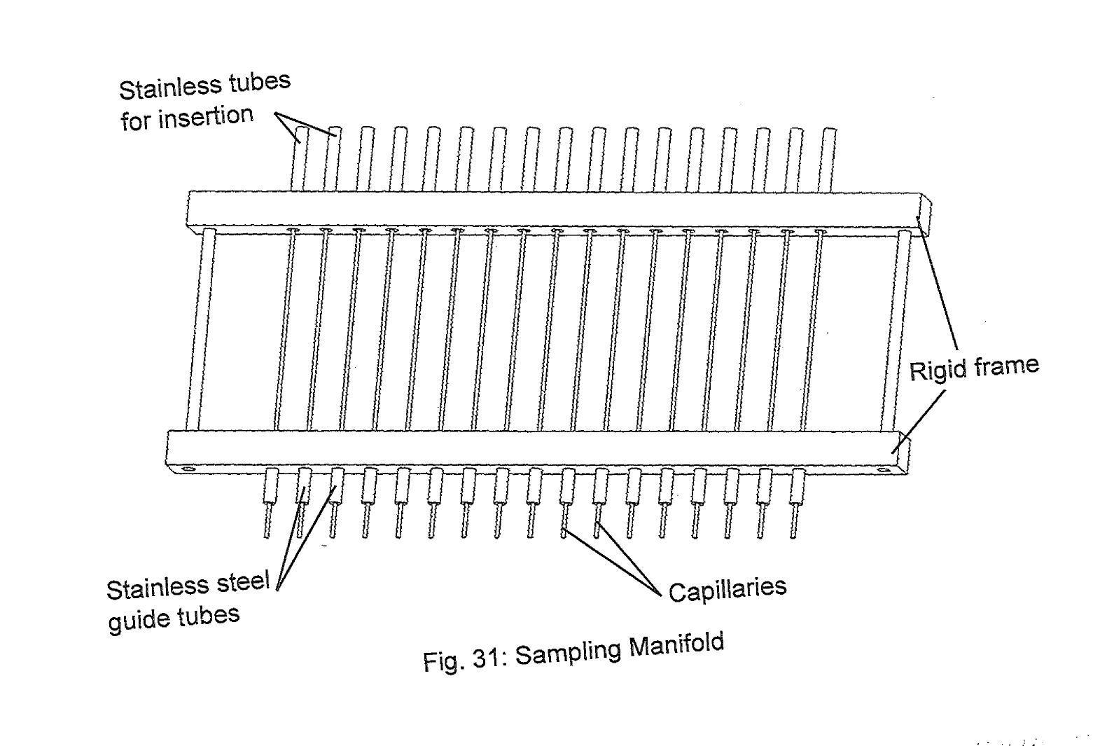

FIG. 30 shows the fluorescence intensity data from stool samples collected from humans along with data from lactoferrin in solution (standard curve values). Spiked stool samples exhibit response that is similar to that of lactoferrin in solution, illustrating the specificity of the antigen/antibody complex and the robustness of the QLISA protocol. The intensity value obtained from the stool sample that does not contain any lactoferrin is essentially the same as the control PMMA capillary of the lactoferrin solution set, indicating the absence of non-specific interaction between the capture antibody and/or the mAb. The lactoferrin-spiked data affirm the expectation that the QLISA bioassay protocol in a full disease model to quantify the presence of lactoferrin in stools and its correlation to clinical disease activity indices.

The multi-sample holder unit is being improved to include automated fluid handling to facilitate capture of MPO (1 nM) and lactoferrin (1 nM) following the QLISA protocol. The unit will have 15 ports in series: 3 for MPO standard solution, 3 titer capillaries for MPO, 3 for lactoferrin standard solution, 3 titer capillaries for lactoferrin, two capillaries for controls (protocol carried out without MPO and lactoferrin) and one capillary for blank. Transporting the capillaries between reservoirs will be carried out manually while fluid flow through the capillaries will be automated. Parameters such as flow rate, temperature and incubation time during MPO and lactoferrin capture and detection will be explored to identify the shortest time required for capturing the antigen. At the end of the capture reaction, capillaries will be detached from the manifold and the fluorescence intensity will be measured using the optical reader designed during the first year funding.

Ultimately kit will measure MPO and lactoferrin in the stool sample to enable the clinician to differentiate IBD from IBS and infectious diarrhea and also follow the diseases response to treatment, monitor remission, relapses or success of anti-inflammatory therapies. Additional markers can be then measured to differentiate ulcerative colitis (UC) from Crohn's disease (CD).

In particular, it is expected that testing using the kit will be able to differentiate IBD from IBS by estimating the amount of lactoferrin present in stool sample, Lactoferrin has been demonstrated to differentiate IBD from IBS at an accuracy of 90%. The concentration of lactoferrin in healthy individuals was estimated to be 3.15+/−1.6 μg/g and in IBD patients 1126.29+/−431.21 μg/g. Quantification of lactoferrin in stool samples will be carried out by a QLIS sandwich assay method similar to that used with MPO. Polyclonal sheep anti-human antibody will be used as the primary antibody to capture lactoferrin in stool samples and QD conjugated mouse anti-human lactoferrin monoclonal antibody will be used as the secondary antibody. MPO, lactoferrin, and calprotectin have been selected as target biomarkers for assessing the degree of inflammation in the case of IBD. Detection and quantification of MPO has been thoroughly studied, and the same strategy is expected apply to similar antigens such as lactoferrin and calprotectin.

A polymer based capillary assay has been developed that relies on the fluorescence intensity of quantum dots to detect picomolar quantities in microliter volumes. Test results have been present of the QLISA device and protocol with respect to Myeloperoxidase (MPO), an antigen that is over expressed in inflammatory conditions. Two different modes of exciting the quantum dots, either using the capillary as the waveguide or using side illumination, were found to be viable, although it was determined that side illumination eliminates problems pertaining to optical alignment and is better suited for a high throughput bioassay. Experimental results show that polymeric capillaries are suitable for optical immunosensor fabrication and that a cost effective biosensor can be fabricated with off the shelf components. The disclosed device has a lowest detection limit of 100 pM towards MPO (˜15 ng/mL). The stability of QDs in the capillaries is found to be affected by the ionic strength of their local environment, and replacing the buffer with a non-polar solution such as glycerol improved their stability.

The advantages of the QLISA method and apparatus for detecting and quantifying MPO, particularly when compared with ELISA, are as follows. First, the volume of sample required for detecting MPO at picomolar concentrations is reduced from 50 μL (96 well plate ELISA set up) to about 1 μL to about 5 μL. Second, the antibody for capturing MPO is covalently bound to the substrate, as opposed to non-specific binding methods used in traditional ELISA or other immunoassay techniques. This creates a robust system, minimizing operator errors and achieving sensitivity and resolution comparable to ELISA. Third, polymethyl methacrylate (PMMA) capillary tubes are used instead of well plates. Fluid handling on well plates is usually carried out by robotic systems which are ideal for diagnostic laboratories but cost prohibitive for hospitals and small clinical laboratories. In contrast, the PMMA capillary tube system utilizes off-the-shelf components for fluid handling, inexpensive high power UV LEDs with extended life (>10,000 hours) for exciting the QDs and modular detection system resulting in a highly adaptable design that could be downsized for small labs in rural areas globally or in ambulatory settings. Fourth, multiplexing in conventional methods (ELISA) would require unique combinations of excitation end emission filters for each antigen under investigation unlike the QLISA method where a single excitation source can be used to excite several QDs and detect multiple antibodies in one sample.

A competitive matrix of available bioassays is shown in below.

| | Parameters | | | |
|---|---|---|---|---|
| | | | Diag- | Sensitivity |
| Products | | False | nostic | and |
| Lab Test (IFT) | Cost ($) | Positives | Value | Specificity |
| Bioxytech EIA-MPO- | 400 | High | Low | Low |
| ANCA (ELISA) | | | | |
| Prometheus ® IBD | 445 | Moderate | Moderate | Low |
| Immuno Concepts - | 369 | High | Low | Low |
| (MPO-ANCA) | | | | |
| ALPCO Catalog | 525 | High | Low | Low |
| Number: 13-CAP- | | | | |
| MPO-110 | | | | |
| QLISA | 50-100 | Low | High | High |
| | (per marker) | | | |

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. An apparatus for detecting a biomarker indicative of an inflammatory condition, the apparatus comprising:
a capillary tube adapted for one or more biomarkers to adhere to an interior surface thereof;
a light source for energizing quantum dots conjugated with the biomarkers within the capillary tube; and
a detection system for detecting and quantifying fluorescent energy emitted by the quantum dots in one or more predetermined wavelength ranges, each wavelength range being correlated to one and only one of the biomarkers.

2. The apparatus of claim 1, wherein the capillary tube comprises a transparent polymer material.

3. The apparatus of claim 1, wherein the capillary tube comprises at least one material selected from the group of polymethyl methacrylate (PMMA), polyvinyl acetate, polycarbonate, and polystyrene.

4. The apparatus of claim 3, further comprising a hypodermic needle connected to an end of the capillary tube for supplying a sample to the capillary tube.

5. The apparatus of claim 3, wherein the capillary tube is supported externally by a glass capillary tube.

6. The apparatus of claim 3, wherein the capillary tube is supported externally by a stainless steel capillary tube.

7. The apparatus of claim 1, further comprising a fluid handling unit adapted to hold multiple capillary tubes and a mechanical positioning system for successively positioning each capillary tube to enable the sample contained therein to be exposed to the light source and visible to the detection system.

8. The apparatus of claim 1, wherein the capillary tube has a volume in the range of about 100 nanoliters to about 1 microliter.

9. The apparatus of claim 1, wherein the light source comprises an LED.

10. The apparatus of claim 9, wherein the LED is an ultraviolet LED.

11. The apparatus of claim 9, wherein the light source comprises an array having a plurality of LEDs.

12. The apparatus of claim 9, wherein the light source further comprises a lens for focusing the LED onto the capillary tube.

13. The apparatus of claim 1, wherein the light source comprises one or more laser diodes.

14. The apparatus of claim 1, wherein the detection system comprises a broadband filter.

15. The apparatus of claim 1, wherein the detection system comprises a photodetector.

16. The apparatus of claim 15, wherein the photodetector is a spectrometer coupled to at least one photomultiplier tube.

17. The apparatus of claim 15, wherein the photodetector is a CCD camera.

18. The apparatus of claim 15, wherein the photodetector is an avalanche photodiode detector.

19. The apparatus of claim 15, wherein the detection system further comprises a fiber optic for transmitting light from the capillary tube to the photodetector.

20. The apparatus of claim 15, wherein the detection system comprises a mirror disposed around at least a portion of the capillary tube for increasing the amount of the fluorescent energy emitted by the quantum dots that can be detected by the photodetector.

21. The apparatus of claim 20, wherein the mirror is selected from the group of a spherical mirror, a cylindrical mirror, and a parabolic mirror.

22. The apparatus of claim 1, wherein the capillary tube is a polymethyl methacrylate (PMMA) capillary tube having a volume of less than about 1.5 microliters; wherein the light source comprises an ultraviolet LED; and wherein the detection system comprises a CCD camera.

23. The apparatus of claim 22, wherein the detection system further comprises a spherical mirror for focusing energy emitted by the quantum dots to the CCD camera.

24. The apparatus of claim 22, wherein a first LED of the light source is directed into an end of the capillary tube and wherein the CCD camera detects energy emitted through the wall of the capillary tube.

25. The apparatus of claim 24, further comprising a second LED of the light source directed into an opposite end of the capillary tube.

26. The apparatus of claim 22, wherein at least one LED of the light source is disposed adjacent to a wall of the capillary tube and wherein the CCD camera detects energy emitted through an end of the capillary tube.

27. The apparatus of claim 1, wherein said biomarker is selected from the group consisting of myeloperoxidase (MPO), IL-1α, TNFα, perinuclear anti-neutrophil cytoplasmic antibody (p-ANCA), anti-Saccharomyces cerevisiae antibody (ASCA), angiotensin converting enzyme, lactoferrin, C-reactive protein (CRP), and calprotectin.

28. The apparatus of claim 1, further comprising a composition for detecting a biomarker in a biological sample contained in the capillary tube, wherein said composition comprises at least one conjugate comprising a quantum dot and an antibody that specifically binds to a biomarker.

29. The apparatus of claim 28, wherein said antibody is bound to a substrate surface.

30. The apparatus of claim 1, wherein the capillary tube is functionalized using NaOH.

31. The apparatus of claim 1, wherein the capillary tube is functionalized using plasma.

32. The apparatus of claim 1, wherein the capillary tube is functionalized using ultraviolet light.

33. A method of diagnosing an inflammatory condition in a subject by detecting a biomarker in a sample, the method comprising:
providing a sample to a capillary tube coated with an antibody, the sample potentially including a biomarker indicative of the inflammatory condition;
contacting the sample with a conjugate comprising a quantum dot and an antibody that specifically binds to the biomarker;
energizing the quantum dot with a light source;
detecting fluorescent emission from the quantum dot; and
correlating the fluorescent emission to the concentration of the biomarker in the sample.

34. The method of claim 33, wherein said biomarker is selected from the group consisting of an enzyme, an adhesion molecule, a cytokine, a protein, a lipid mediator, an immune response mediator, and a growth factor.

35. The method of claim 33, wherein said biomarker is selected from the group consisting of myeloperoxidase (MPO), IL-1α, TNFα, perinuclear anti-neutrophil cytoplasmic antibody (p-ANCA), anti-Saccharomyces eerevisiae antibody (ASCA), angiotensin converting enzyme, lactoferrin, C-reactive protein (CRP), and calprotectin.

36. The method of claim 33, wherein said inflammatory condition comprises at least one inflammatory disease selected from the group consisting of inflammatory bowel disease, ulcerative colitis, Crohn's disease, stroke, myocarditis, cardiovascular disease, acute coronary syndromes, acute myocardial infarction, pericarditis, periodontal disease, cancer, Alzheimer's disease, and autoimmune diseases.

37. A method of stabilizing the fluorescence of quantum dots over time comprising exposing the quantum dots to a fluorescence stabilizing medium.

38. The method of claim 37, wherein the fluorescence stabilizing medium is a solution having a low ionic strength.

39. The method of claim 37, wherein the fluorescence stabilizing medium is a solution having a pH greater than or equal to about 7.0.

40. The method of claim 39, wherein the solution has a pH greater than or equal to about 8.0.

41. The method of claim 37, wherein the fluorescence stabilizing medium comprises water-soluble free radical quenchers.

42. The method of claim 37, wherein the fluorescence stabilizing medium comprises TrisPro and an amount of water-soluble vitamin E.

43. The method of claim 41, wherein the amount of vitamin E is at least about 0.001% of said medium.

44. The method of claim 37, wherein the quantum dots each comprise a CdSe core and a ZnS protective layer.
